SEQUENCE LISTING (18 sequences)

SEQ ID NO: 1 - DNA, 23 bp, artificial sequence
  synthetic primer
atgcgaccctcagggaccgcgag

SEQ ID NO: 2 - DNA, 29 bp, artificial sequence
  synthetic primer
ccgctcgaggatgttatgttcaggccgac

SEQ ID NO: 3 - DNA, 28 bp, artificial sequence
  synthetic primer
cgcaagcttcgggagagccggagcgagc

SEQ ID NO: 4 - DNA, 28 bp, artificial sequence
  synthetic primer
ccgctcgaggccttgcagctgttttcac

SEQ ID NO: 5 - DNA, 28 bp, artificial sequence
  synthetic primer
cgcaagcttcgggagagccggagcgagc

SEQ ID NO: 6 - DNA, 26 bp, artificial sequence
  synthetic primer
cgcgttaacgatgttatgttcaggct

SEQ ID NO: 7 - DNA, 54 bp, artificial sequence
  synthetic primer
agcgcggcgccgtggcaggttccgtctctttcttggcaggccgttaccaggccg

SEQ ID NO: 8 - DNA, 48 bp, artificial sequence
  synthetic primer
ctggtaacggcctgccaagaaagagacggaacctgccacggcgccgcg

SEQ ID NO: 9 - DNA, 54 bp, artificial sequence
  synthetic primer
cttcatccgctagcccaaaaccgcgtcagctgggacacaggcccctctagacgc

SEQ ID NO: 10 - DNA, 60 bp, artificial sequence
  synthetic primer
ccgcgtctagaggggcctgtgtcccagctgacgcggttttgggctagcggatgaagcggc

SEQ ID NO: 11 - DNA, 18 bp, artificial sequence
  synthetic primer
agcgcggcgccgtggcag

SEQ ID NO: 12 - DNA, 18 bp, artificial sequence
  synthetic primer
ccgcgtctagaggggcct

SEQ ID NO: 13 - protein, 501 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKA

SEQ ID NO: 14 - protein, 478 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLPWQVPSLSWQAVTRPLHPLAQNRVSWDTGP

SEQ ID NO: 15 - protein, 447 aa, Rattus norvegicus
MRPSGTARTTLLVLLTALCAAGGALEEKKVCQGTSNRLTQLGTFEDHFLSLQRMYNNCEVVLGNLEITYVQRNYDLSFLKTIQEVAGYFLIALNTVERIPSEDLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPQKSDCLVCQKFQDEATCKDTCPPLMLYNPTTYQMDVNPEGKYSFGATCVKNCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGSLLIQAWPENWTDLHAFENLEIIRGRTKQHGQFSLAVVGLNITS

SEQ ID NO: 16 - protein, 478 aa, Rattus norvegicus
MRPSGTARTTLLVLLTALCAAGGALEEKKVCQGTSNRLTQLGTFEDHFLSLQRMYNNCEVVLGNLEITYVQRNYDLSFLKTIQEVAGYFLIALNTVERIPSEDLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPQKSDCLVCQKFQDEATCKDTCPPLMLYNPTTYQMDVNPEGKYSFGATCVKNCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGSLLIQAWPENWTDLHAFENLEIIRGRTKQHGQFSLAVVGLNITSLPWQVPSLSWQAVTRPLHPLAQNRVSWDTGP

SEQ ID NO: 17 - DNA, 1434 bp, Artificial Sequence
  based on human EGFR
atgcgtccttctggtactgctggtgctgctcttcttgctcttcttgctgctctttgtcctgcttctcgtgctcttgaagaaaaaaaagtttgtcaaggtacttctaataaacttactcaacttggtacttttgaagatcattttctttctcttcaacgtatgtttaataattgtgaagttgttcttggtaatcttgaaattacttatgttcaacgtaattatgatctttcttttcttaaaactattcaagaagttgctggttatgttcttattgctcttaatactgttgaacgtattcctcttgaaaatcttcaaattattcgtggtaatatgtattatgaaaattcttatgctcttgctgttctttctaattatgatgctaataaaactggtcttaaagaacttcctatgcgtaatcttcaagaaattcttcatggtgctgttcgtttttctaataatcctgctctttgtaatgttgaatctattcaatggcgtgatattgtttcttctgattttctttctaatatgtctatggattttcaaaatcatcttggttcttgtcaaaaatgtgatccttcttgtcctaatggttcttgttggggtgctggtgaagaaaattgtcaaaaacttactaaaattatttgtgctcaacaatgttctggtcgttgtcgtggtaaatctccttctgattgttgtcataatcaatgtgctgctggttgtactggtcctcgtgaatctgattgtcttgtttgtcgtaaatttcgtgatgaagctacttgtaaagatacttgtcctcctcttatgctttataatcctactacttatcaaatggatgttaatcctgaaggtaaatattcttttggtgctacttgtgttaaaaaatgtcctcgtaattatgttgttactgatcatggttcttgtgttcgtgcttgtggtgctgattcttatgaaatggaagaagatggtgttcgtaaatgtaaaaaatgtgaaggtccttgtcgtaaagtttgtaatggtattggtattggtgaatttaaagattctctttctattaatgctactaatattaaacattttaaaaattgtacttctatttctggtgatcttcatattcttcctgttgcttttcgtggtgattcttttactcatactcctcctcttgatcctcaagaacttgatattcttaaaactgttaaagaaattactggttttcttcttattcaagcttggcctgaaaatcgtactgatcttcatgcttttgaaaatcttgaaattattcgtggtcgtactaaacaacatggtcaattttctcttgctgttgtttctcttaatattacttctcttccttggcaagttccttctctttcttggcaagctgttactcgtcctcttcatcctcttgctcaaaatcgtgtttcttgggatactggtcct

SEQ ID NO: 18 - DNA, 1434 bp, Artificial Sequence
  based on rat ERRP
atgcgtccttctggtactgctcgtactactcttcttgttcttcttactgctctttgtgctgctggtggtgctcttgaagaaaaaaaagtttgtcaaggtacttctaatcgtcttactcaacttggtacttttgaagatcattttctttctcttcaacgtatgtataataattgtgaagttgttcttggtaatcttgaaattacttatgttcaacgtaattatgatctttcttttcttaaaactattcaagaagttgctggttattttcttattgctcttaatactgttgaacgtattccttctgaagatcttcaaattattcgtggtaatgctctttatgaaaatacttatgctcttgctattctttctaattatggtactaatcgtactggtcttcgtgaacttcctatgcgtaatcttcaagaaattcttattggtgctgttcgtttttctaataatcctattctttgtaatatggatactattcaatggcgtgatattgttcaaaatgtttttatgtctaatatgtctatggatcttcaatctcatccttcttcttgtcctaaatgtgatccttcttgtcctaatggttcttgttggggtggtggtgaagaaaattgtcaaaaacttactaaaattatttgtgctcaacaatgttctcatcgttgtcgtggtcgttctccttctgattgttgtcataatcaatgtgctgctggttgtactggtcctcaaaaatctgattgtcttgtttgtcaaaaatttcaagatgaagctacttgtaaagatacttgtcctcctcttatgctttataatcctactacttatcaaatggatgttaatcctgaaggtaaatattcttttggtgctacttgtgttaaaaattgtcctcgtaattatgttgttactgatcatggttcttgtgttcgtgcttgtggtcctgattattatgaagttgaagaagatggtattcgtaaatgtaaaaaatgtgatggtccttgtcgtaaagtttgtaatggtattggtattggtgaatttaaagatactctttctattaatgctactaatattaaacattttaaatattgtactgctatttctggtgatcttcatattcttcctgttgcttttaaaggtgattcttttactcgtactcctcctcttgatcctcgtgaacttgaaattcttaaaactgttaaagaaattactggttctcttcttattcaagcttggcctgaaaattggactgatcttcatgcttttgaaaatcttgaaattattcgtggtcgtactaaacaacatggtcaattttctcttgctgttgttggtcttaatattacttctcttccttggcaagttccttctctttcttggcaagctgttactcgtcctcttcatcctcttgctcaaaatcgtgtttcttgggatactggtcct

(19) United States
(12) Patent Application Publication
(10) Pub. No.: US 2012/0121589 A1
(43) Pub. Date: May 17, 2012

(54) MODIFIED EGFR ECTODOMAIN

(75) Inventors: Adhip P. N. Majumdar, Ferndale, MI (US); Arun K. Rishi, Troy, MI (US); Yingjie Yu, Canton, MI (US)

(73) Assignee: WAYNE STATE UNIVERSITY, DETROIT, MI (US)

(21) Appl. No.: 13/383,536

(22) PCT Filed: Jul. 13, 2010
(86) PCT No.: PCT/US10/41838

§ 371 Date: Jan. 11, 2012

Related U.S. Application Data
(60) Provisional application No. 61/225,084, filed on Jul. 13, 2009.

(51) Int. Cl. A61K 39/395; A61K 38/02; A61P 35/02; C12N 15/63; C12N 5/071; A61P 35/00; C07K 14/71; C12N 15/54

(52) U.S. Cl. 424/133.1; 530/350; 514/19.3; 536/23.2; 435/320.1; 435/375; 514/19.4; 514/19.6

(57) ABSTRACT

A protein that attenuates EGFR and/or EGFR family members comprises a modified EGFR ectodomain. The protein inhibits signaling via the EGFR and/or EGFR family members. The protein includes a portion of the EGFR (or EGFR family member) and the “U” region epitope of EGFR related protein (ERRP), wherein the portion of the EGFR is operable to bind a ligand of EGFR. Also included are nucleic acids encoding such proteins. Attenuating EGFR signaling can include inhibiting the EGFR and/or EGFR family members and to provide antiproliferative activity. The present proteins and expression of nucleic acids encoding these proteins can regulate cellular growth and can be used to treat tumors and cancerous cells that express one or more of the EGFR and EGFR family members.

### INTRODUCTION

The present technology relates to modified epidermal growth factor receptors (EGFR), including uses thereof to attenuate EGFR signaling.

Members of the receptor tyrosine kinase family are frequently implicated in experimental models of epithelial cell neoplasia as well as in human cancers. There is increasing evidence to support the concept that the malignant behavior of some tumors is sustained by deregulated activation of certain growth factor receptors. Such deregulation may be due to structural alterations of the receptor itself or to the establishment of an autocrine loop, whereby cells produce growth factors that stimulate their own growth.

Growth factor receptors with intrinsic tyrosine kinase activity are activated following the binding of growth factors. One of the best studied receptor signaling systems from this family of receptors is that controlled by the EGF-receptor (EGFR), whose expression and enzyme activity have been linked to a number of malignancies, including cancers of the gastrointestinal tract. EGFR and its ligand TGF-α, a structural and functional analogue of EGF, are overexpressed in preneoplastic and epithelial cells. However, it is becoming increasingly evident that most solid tumors not only express EGFR but also express other family members that include ErbB-2/HER-2, ErbB-3/HER-3 and ErbB-4/HER-4.

The vast number of patients with tumors expressing EGFR and/or family member(s) provides a logical setting for the use of signal transduction inhibitors. Indeed, several drugs such as cetuximab (Erbitux™), trastuzumab (Herceptin™), gefitinib (Iressa™) and erlotinib (Tarceva™) that target either EGFR or HER-2 have been developed, but with limited success. This may partly be the result of the fact that most solid tumors express more than one member of the EGFR family, and coexpression of multiple EGFR family members leads to an enhanced transforming potential and worsened prognosis. However, most conventional EGFR targeted therapeutics target only a specific member of the EGFR family. Therefore, identification of inhibitors that target multiple members of the EGFR family is likely to provide a therapeutic benefit to a broad range of the patient population.

Many solid tumors, including breast cancers, show increased activation of several growth factor receptors, specifically EGFR and its family members as well as c-Src, a nonreceptor tyrosine kinase that promotes proliferation, inhibits apoptosis, and induces metastasis. Breast cancer is the second leading cause of cancer-related deaths among females, next only to lung cancer. It is a complex disease. Based on transcriptional profiling, breast cancer is currently identified in five distinct subtypes: luminal A and B, normal breast-like, HER-2 over-expressing, and basal-like.

Basal-like breast cancer, which shows the absence of hormone receptors (estrogen receptor and progesterone receptor) without amplification of HER-2, is referred to as triple-negative breast cancer. As a group, basal-like cancers comprise about 80% of triple-negative cancers. At present, there is controversy about the classification of basal and triple-negative breast cancers. For the sake of simplicity, these two terms are frequently used interchangeably. Triple-negative breast cancer is found to be more common among African-American and BRCA1 mutation carriers. It is associated with aggressive histology, poor prognosis, and unresponsiveness to usual endocrine therapies, highlighting the need for new therapeutics/strategies.

### SUMMARY

The present technology is drawn to methods and compositions that include polypeptides comprising modified EGFRs and modified EGFR family members that attenuate signaling via the EGFR and EGFR family members. These polypeptides include inhibitors of the EGFR and EGFR family members. Polypeptides are provided that comprise a portion of the EGFR (or EGFR family member) and the U-region epitope of EGFR related protein (ERRP), wherein the protein is operable to bind a ligand of EGFR. The portion of the EGFR or EGFR family member that binds a ligand of EGFR may be a portion of the extracellular domain (ectodomain) of EGFR. For example, the polypeptide may comprise an epidermal growth factor receptor (EGFR) ectodomain coupled at the C-terminus to a U-region. The extracellular domain may comprise domains I, II, and III of the EGFR. In some embodiments, the protein comprises SEQ ID NO: 14 or SEQ ID NO: 16. Also included are variants of the aforementioned proteins, for example, variants comprising an amino acid sequence that is at least 80% identical to SEQ ID NO: 14 or SEQ ID NO: 16 and up to at least 98% identical to SEQ ID NO: 14 or SEQ ID NO: 16.

The present technology further includes nucleic acids encoding the aforementioned polypeptides, such as a nucleic acid encoding a polypeptide comprising an epidermal growth factor receptor (EGFR) ectodomain coupled at the C-terminus to a U-region. Nucleic acids include single and double stranded form of DNA and RNA. Recombinant DNA, expression cassettes, and expression vectors encoding such polypeptides are also included.

The present technology provides methods of using proteins comprising modified EGFRs and modified EGFR family members, or methods expressing nucleic acids encoding such proteins, in order to attenuate signaling via the EGFR and/or EGFR family members. Attenuating EGFR signaling can include inhibiting the EGFR and/or EGFR family members and can provide antiproliferative activity. For example, the present proteins and expression of nucleic acids encoding these proteins can function to regulate cellular growth and may even prevent cellular growth. In some cases, tumors and cancerous cells that express one or more EGFRs and/or EGFR family members can be treated using the present proteins. The present proteins can be more effective than other treatments since multiple members of the EGFR family can be targeted in comparison to therapies that only target a specific member of the EGFR family.

Proteins comprising modified EGFRs and modified EGFR family members can also be used in combination with various signal transduction inhibitors, such as tyrosine kinase inhibitors including non-receptor tyrosine kinase inhibitors. For example, one or more modified EGFRs, family members, homologues, or recombinant constructs comprising an EGFR ectodomain coupled at the C-terminus to a U-region may be used with one or more signal transduction inhibitors, including cetuximab (Erbitux™), trastuzumab (Herceptin™), gefitinib (Iressa™), erlotinib (Tarceva™), dasatinib (Sprycel™), imatinib, bevacizumab, sorafenib, and sunitinib.

The present polypeptides comprising an epidermal growth factor receptor (EGFR) ectodomain coupled at the C-terminus to a U-region and combinations of the polypeptides with one or more signal transduction inhibitors can be used to treat various conditions, including: breast cancer, chronic myeloid leukemia (CML), gastrointestinal stromal tumors (GIST), non-small cell lung cancer (NSCLC), colorectal cancer (CRC), pancreatic cancer, renal cell cancer, and head and neck cancer.

### DRAWINGS

The present technology will become more fully understood from the detailed description and the accompanying drawings.

FIG. 1 is a schematic representation of one embodiment of a recombinant DNA encoding a fusion of a “U” region with a human EGFR fragment;

FIG. 2 is a schematic representation of human EGFR (hEGFR), truncated EGFRs of rat and human origin (upper panel), and production of these peptides in the absence (−) or presence (+) of CuSO₄ (lower panel);

FIG. 3 illustrates the effects of recombinant protein from truncated EGFR plasmids (ERRP, hEGFR-501, hEGFR-448+U and rEGFR-447) on the growth (as determined by MTT) of colon cancer HCT-116 cells;

FIG. 4 illustrates the effects of recombinant proteins from various truncated EGFR plasmids [ERRP, hEGFR-501, hEGFR-448+U (EBIP) and rEGFR-447] on inhibition of TGF-α-induced phosphorylation of EGFR, with β-actin used as a loading control;

FIG. 5 illustrates the effects of recombinant proteins from truncated EGFR plasmids [ERRP, hEGFR-501, hEGFR-448+U (EBIP) and rEGFR-447] on inhibition of TGF-α-induced phosphorylation of Src in colon cancer HCT-116 cells;

FIG. 6 illustrates the effects of recombinant proteins from different truncated EGFR plasmids [ERRP, hEGFR-501, hEGFR-448+U (EBIP) and rEGFR-447] on inhibition of TGF-α-induced phosphorylation of ERKs in colon cancer HCT-116 cells, with α-tubulin used as a loading control;

FIG. 7 illustrates the effects of recombinant proteins from different truncated EGFR plasmids [ERRP, hEGFR-501, hEGFR-447+U and rEGFR-447] on the growth (as determined by MTT) of breast cancer SKBR-3 and MDA-MB-453 cells;

FIG. 8 illustrates the effects of recombinant proteins from different truncated EGFR plasmids [ERRP, hEGFR-501, hEGFR-448+U (EBIP) and rEGFR-447] on inhibition of heregulin-induced phosphorylation of HER-2 and HER-3, with α-tubulin used as loading control;

FIG. 9 illustrates treatment of SKBR3 palpable xenograft tumor in SCID mice with hEGFR-448+U (EBIP);

FIGS. 10(a)-(c) illustrate DNA coding sequences for the amino acid sequence of human EGFR ectodomain containing amino acids 1-448 plus the 30 amino acid U-region; and

FIGS. 11(a)-(c) illustrate DNA coding sequences for the amino acid sequence of rat ERRP containing amino acids 1-478.

FIG. 12: Panel A is a schematic representation of (i) full-length hEGFR and four different plasmid constructs of (ii) rat ERRP (amino acids 1-478), (iii) rat EGFR ectodomain (ERRP without U region; referred to as ERRP-448), (iv) hEGFR ectodomain (referred to as hEGFR-501), and (v) hEGFR ectodomain fused with U region (referred to as hEGFR-448+U or EBIP). Panel B shows synthesis of recombinant proteins by Drosophila S2 cells in the absence (−) or presence (+) of CuSO₄ as determined by Western blot analysis of the cell lysates. Recombinant proteins containing both His and V5 tags were purified using His tag and immunoblotted with V5 antibody. Panel C shows Western blot analysis of EBIP localization in response to TGF-α induction of breast cancer cells. After 8 h of incubation with EBIP, MDA-MB-468 cells, which were serum starved, were induced with TGF-α. The cell lysates were immunoprecipitated with EGFR antibodies overnight and the immunoprecipitates were subsequently subjected to Western blot analysis with V5 antibody for EBIP detection. Panel D shows inhibition of growth of MDA-MB-468 cells in response to immunoaffinity-purified EBIP and ERRP.

FIG. 13: Panel A is a Western blot showing the levels of EGFR, HER-2, and HER-3 in four different human breast cancer cells. Effects of EBIP and/or dasatinib on growth of human breast cancer cells expressing varying levels of EGFR members. Growth was determined by MTT assay after 48 h of exposure to increasing doses of dasatinib (Panel B) and EBIP (Panel C). Panel D shows combined therapy of dasatinib (Das; 1.0 μmol/L) and EBIP (2.5 μg/mL purified protein) in human breast cancers. *, P<0.05, compared with individual drugs. Columns, mean of six observations; bars, SD.

FIG. 14 illustrates typical dose-response curves for EBIP and/or dasatinib in MDA-MB-231 (A) and MDA-MB-468 (B) cells produced by fixed-ratio method. Fraction of breast cancer cells affected by different combination of dasatinib and EBIP (fixed ratio) is higher than either agent alone. Fa represents the fraction of cells that is growth inhibited in response to dasatinib and/or EBIP. This is calculated as 1−fraction of surviving cells. Fa values for each treatment were used to conduct synergy analysis by CalcuSyn software as described herein.

FIG. 15: Panel A shows effects of EBIP and/or dasatinib on different aspects of growth of MDA-MB-468 breast cancer cells on induction of apoptosis as determined by DNA fragmentation ELISA assay in response to EBIP and/or dasatinib. †, P<0.05, compared with control. Panel B shows induction of early apoptosis as determined by acridine orange staining method. Cells were coincubated with combination of dasatinib (1 mmol/L) and EBIP (2.5 μg/mL) and specific inhibitors of caspase-8 or caspase-9. *, P<0.05, compared with combination therapy without caspase inhibitors. Panel C shows EGFR signaling and the levels of tyrosine-phosphorylated forms of EGFR and c-Src and their downstream signaling molecules in response to EBIP and/or dasatinib, as determined by Western blot analysis. The experiment was repeated at least thrice. Panel D shows tyrosine kinase activity of EGFR. Lysates from cells treated with respective drugs were immunoprecipitated with EGFR. Protein-Sepharose bead complexes were assayed for kinase activity using ELISA-based assay. *, P<0.05, compared with EBIP; ¶, P<0.05, compared with dasatinib alone. Columns, mean of four observations; bars, SD.

FIG. 16 illustrates a preclinical efficacy trial of EBIP and/or dasatinib in MDA-MB-468 xenografts of SCID mice. Once palpable tumors developed (day 7), the treatment was initiated with EBIP and/or dasatinib. EBIP (25 μg/animal) was administered s.c. (away from tumor), whereas dasatinib (10 mg/kg) was given orally (gavage), every other day for 16 d. Changes in tumor weight were recorded during the 55-d experimental period. Points, mean; bars, SE.

FIG. 17 illustrates immunohistochemical demonstration of EBIP in the tumor remnants from mice sacrificed at the end of the experimental period. EBIP was detected in the tumor remnants by V5 antibody staining Panel A, left, shows a photomicrograph from a vehicle-treated animal; right, shows a photomicrograph from an EBIP-treated animal. Panel B shows changes in number of apoptotic cells as determined by TUNEL staining. Panel C shows levels of uncleaved PARP as determined by Western blot analysis. Protein extracts were made from the tumor remnants.

FIG. 18 graphically depicts the growth-inhibitory properties of hEGFR-501, hEGFR-448+U, ERRP, and rEGFR-447 (lacked U region) in colon cancer HCT-116 cells.

FIG. 19 illustrates that ERRP and EBIP are able to inhibit heregulin-induced activation of HER-2 and HER-3 in MDA-MB-453 breast cancer cells while neither rEGFR-447 nor hEGFR-501 re effective.

FIG. 20 shows that tumor remnants from mice treated with EBIP or EBIP+dasatinib have no detectable immunoreactivity for phospho-EGFR, whereas those from the controls and dasatinib-treated mice show the presence of phospho-EGFR.

It should be noted that the figures set forth herein are intended to exemplify the general characteristics of compositions, systems and methods among those of the present technology, for the purpose of the description of specific embodiments. These figures may not precisely reflect the characteristics of any given embodiment, and are not necessarily intended to define or limit specific embodiments within the scope of this technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present technology is drawn to a protein comprising at least a portion of the ectodomain of the epidermal growth factor receptor (EGFR) and a carboxy-terminus epitope of about 30-amino acids (“U” region epitope) from the EGFR related protein (ERRP). Nucleic acids encoding and/or expressing such proteins are also included. Uses of these proteins and nucleic acids include attenuation of EGFR and EGFR family member signaling, inhibition of EGFR and EGFR family members, antiproliferative treatments, and cancer treatments.

EGFR is a 170 kDa transmembrane glycoprotein protein with intrinsic tyrosine kinase activity that binds the EGF family of peptides. The EGFR possesses three functional domains that include an extracellular domain (also known as the ectodomain), transmembrane domain, and a cytoplasmic domain. Ligand binding to the extracellular domain of EGFR leads to dimerization and activation of the receptor's intrinsic tyrosine kinase, located in the cytoplasmic domain, triggering a complex array of enzymatic and biological events that can lead to cell proliferation and differentiation.

Overexpression of EGFR has been associated with many malignancies, including cancers of the stomach and colon. Evidence is accumulating that shows that malignant as well as certain normal cells also produce other forms of EGFR and/or EGFR family members. For example, A431 human epidermoid carcinoma cells produce a truncated EGFR that encodes a 2.8 kb mRNA transcript that is thought to be the result of gene rearrangement in chromosome 7. Likewise, normal rat liver produces a 2.7 kb mRNA transcript whose 5′, but not 3′, sequences show 100% homology with the external domain of the full-length rat EGFR. These observations suggest that this truncated form of EGFR mRNA, whose protein product is also secreted from the cell, is generated from alternative splicing of the primary EGFR transcript. A shorter 1.8 kb alternative transcript from the human EGFR, which also produces a secreted product, has been isolated from a human placental cDNA library.

Given the fact that ligand binding and subsequent homo- or hetero-dimerization of EGFRs are essential for activation, any intervention that affects these processes may have a profound effect on the signal transduction pathways induced by EGFR and/or its family members. For example, transfection of an extracellular fragment of EGFR or kinase-negative dominant mutants of EGFR results in inhibition of EGFR phosphorylation and of anchorage-dependent and/or independent growth. Additionally, it has been demonstrated that infection of pancreatic cancer cell lines (ASPC-1, COLO-357, PANC-1, and T3M4) with an adenoviral vector encoding a truncated EGFR attenuates EGF and HB-EGF-dependent cell growth, tyrosine phosphorylation of EGFR family members, and phosphorylation of ERKs, JNKs and p38 as well as activating transcription factor 2. Taken together, these data suggest that truncated EGFR generated by molecular biology manipulations modulates the EGFR signaling process and plays a role in regulating cellular growth.

In addition, an ectodomain of EGFR known as ERRP (EGFR Related Protein; U.S. Pat. No. 6,399,743, Majumdar, issued Jun. 4, 2002 and U.S. Pat. No. 6,582,934, Majumdar, issued Jun. 24, 2003; GenBank accession #AF187818; all of which are incorporated herein by reference) has growth inhibitory properties. ERRP is isolated from the rat gastro-duodenal mucosa and subsequently characterized as a pan-erbB inhibitor that targets multiple members of the EGFR family. ERRP possesses 3 of the 4 extracellular subdomains of EGFR and may exert its growth inhibitory properties by competing for the ligands of EGFR thereby attenuating their activation. A truncated EGFR with only 3 of the 4 extracellular subdomains is reported to bind EGF and TGF-α with at least ten-fold higher affinity than the full-length extracellular domain of EGFR (Garrett T P J, McKern N M, Lou M, Ellman T J, Adams T E, Lovrecz G O, Zhu H-J, Walker F, Frenkel M J, Hoyne P A, Jorissen R N, Nice E C, Burgess A W, Ward C W: Crystal structure of a truncated epidemial growth factor receptor extracellular domain bound to transforming growth factor α. Cell 110: 763-773, 2002). Since ERRP lacks most of the extracellular domain IV, ERRP may also be very effective in preferentially binding/sequestering ligands of EGFR. In view of the fact that ERRP possesses extracellular subdomains I to III, but lacks transmembrane and cytoplasmic domains, ERRP inhibition of EGFR function further corroborates with the EGFR inhibitory properties of such subdomains.

Without limiting the compositions, methods or operation of the present technology, in various embodiments the carboxy-terminus epitope of 30-amino acids, referred to herein as the “U” region, plays an important role in ERRP-dependent attenuation of EGFRs and subsequent cell growth. Loss of this epitope significantly affects the growth inhibitory properties of ERRP. To demonstrate this effect, expression plasmids of truncated human EGFR ectodomain with or without the “U” region epitope of ERRP can be generated and their growth inhibitory properties compared with that of ERRP.

Expression constructs are generated as follows.

Rat EGFR Ectodomain, (ERRP without “U” region; referred to herein as ERRP-447): Rat EGFR sequences corresponding to ERRP (amino acids 1-447) are amplified using the polymerase chain reaction (PCR) and the following forward and reverse primers:

Forward:
(SEQ ID NO: 1)
5′-ATGCGACCCTCAGGGACCGCGAG-3′
Reverse:
(SEQ ID NO: 2)
5′-CCGCTCGAGGATGTTATGTTCAGGCCGAC-3′

The PCR product is cut with XhoI restriction enzymes and subcloned into EcoRV+XhoI cut pMT/His-V-5B vector (Invitrogen, Carlsbad, Calif.) to obtain a recombinant plasmid for expression of V-5-His-tagged rat EGFR ectodomain sequences.

Human EGFR Ectodomain (referred to herein as hEGFR-501): Human EGFR sequences from amino acids 1 to 501 are PCR amplified using the following forward and reverse primers:

Forward:
(SEQ ID NO: 3)
5′-CGCAAGCTTCGGGAGAGCCGGAGCGAGC-3′
Reverse:
(SEQ ID NO: 4)
5′-CCGCTCGAGGCCTTGCAGCTGTTTTCAC-3′

Position 501 is selected for truncation since this truncated ectodomain of human EGFR (hEGFR) binds EGFR ligands (e.g., EGF and TGF-α) with 13- to 14-fold higher affinity than the full-length EGFR ectodomain. The PCR product is cut with XhoI restriction enzyme and subcloned into EcoRV+XhoI cut pMT/His-V-5B vector to obtain a plasmid for expression of His-V5-tagged hEGFR-501 ectodomain sequences.

The cloning of Human EGFR Ectodomain fused with “U” Region (referred to herein as hEGFR-448+U and as ErbB Inhibitory Protein (EBIP)) is performed using steps (i)-(iv) as follows.

(i) Human EGFR sequences from amino acids 1 to 448 are first PCR amplified using the following forward and reverse primers:

Forward:
(SEQ ID NO: 5)
5′-CGCAAGCTTCGGGAGAGCCGGAGCGAGC-3′;
and
Reverse:
(SEQ ID NO: 6)
5′-CGCGTTAACGATGTTATGTTCAGGCT-3′.

This PCR product is digested with HindIII and HpaI, and gel purified for subsequent 3-way ligation.

(ii) The “U” region epitope from ERRP is synthesized as oligonucleotides with codons optimized for human expression. The oligonucleotides are as follows.

hERRP-U region Oligo-1:
(SEQ ID NO: 7)
5′-
AGCGCGGCGCCGTGGCAGGTTCCGTCTCTTTCTTGGCAGGCCGTTACCAG
GCCG-3′
hERRP-U region Oligo-2:
(SEQ ID NO: 8)
5′-CTGGTAACGGCCTGCCAAGAAAGAGACGGAACCTGCCACGGCGCCGC
G-3′
hERRP-U region Oligo-3:
(SEQ ID NO: 9)
5′-
CTTCATCCGCTAGCCCAAAACCGCGTCAGCTGGGACACAGGCCCCTCTAG
ACGC-3′
hERRP-U region Oligo-4:
(SEQ ID NO: 10)
5′-
CCGCGTCTAGAGGGGCCTGTGTCCCAGCTGACGCGGTTTTGGGCTAGCGG
ATGAAGCGGC-3′

The oligonucleotides are phosphorylated at the respective 5′ ends using T4 polynucleotide kinase, and annealed as follows: oligos 1+2; and 3+4. The annealed products are ligated to obtain a contiguous “U” region sequence. This double stranded “U” region sequence is then utilized as a template in PCR using the following forward and reverse primers:

| (SEQ ID NO: 11) | |
|---|---|
| Forward: | 5′-AGCGCGGCGCCGTGGCAG-3′; |
| and | |
| (SEQ ID NO: 12) | |
| Reverse: | 5′-CCGCGTCTAGAGGGGCCT-3′. |

The PCR product is cut with a combination of SfoI and XbaI restriction enzymes and the product gel purified.

(iii) The PCR amplified products from Steps (i) and (ii) are ligated into HindIII plus XbaI cut vector plasmid pcDNA-3/myc-His-A to obtain a recombinant plasmid for expression of Myc-His-tagged hEGFR+U protein.

(iv) The cDNA insert of the recombinant plasmid from Step (iii) above is PCR amplified using the forward primer from Step-i and the reverse primer from Step (ii). The PCR product is then cut with XbaI and ligated into EcoRV plus XbaI cut pMT/V5-HisA vector to obtain a construct for expression of V5/His-tagged hEGFR+U protein.

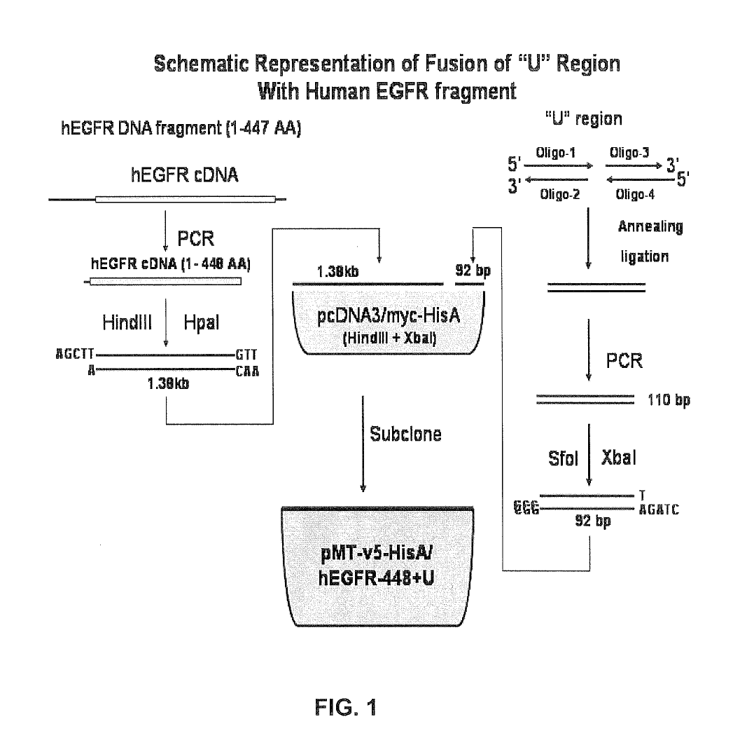

All the expression constructs are sequenced to confirm the validity of the inserts. A schematic representation of the construction of human EGFR-448+“U” region of ERRP is shown in FIG. 1.

Rat ERRP: described in U.S. Pat. No. 6,399,743, Majumdar, issued Jun. 4, 2002; U.S. Pat. No. 6,582,934, Majumdar, issued Jun. 24, 2003; and GenBank accession # AF187818, which are incorporated herein by reference. Rat ERRP is composed of 478 amino acids.

Recombinant fusion proteins are generated as follows. The drosophila expression system can be used to generate the respective fusion proteins. In the first instance, each pMT/V5-His-recombinant plasmid is transfected into S2 cells. Expression of the recombinant proteins is induced with 0.5 mM CuS0₄ and confirmed by Western-blot analysis of cell lysates in conjunction with anti-V5 antibodies (Invitrogen, Carlsbad, Calif.). Stable hygromycin-resistant S2 cells for expression of each of the fusion proteins are generated, followed by immunoaffinity purification as described in Marciniak et al., Epidermal growth factor receptor related protein (ERRP): a potential therapeutic agent for colorectal cancer, Gastroenterology 124: 1337-1347 (2003).

In sum, the four expression constructs include the following proteins:

(a) human EGFR ectodomain that includes amino acids 1-501 but lacks the “U” region (hEGFR-1-501) (SEQ ID NO: 13);

(b) human EGFR ectodomain that includes amino acids 1-448 plus the “U” region (hEGFR448+U or EBIP) (SEQ ID NO: 14);

(c) rat ERRP that includes amino acids 1-447 but lacks the “U” region (rEGFR-447) (SEQ ID NO: 15); and

(d) rat ERRP that includes amino acids 1-478 and includes the “U” region (ERRP) (SEQ ID NO: 16).

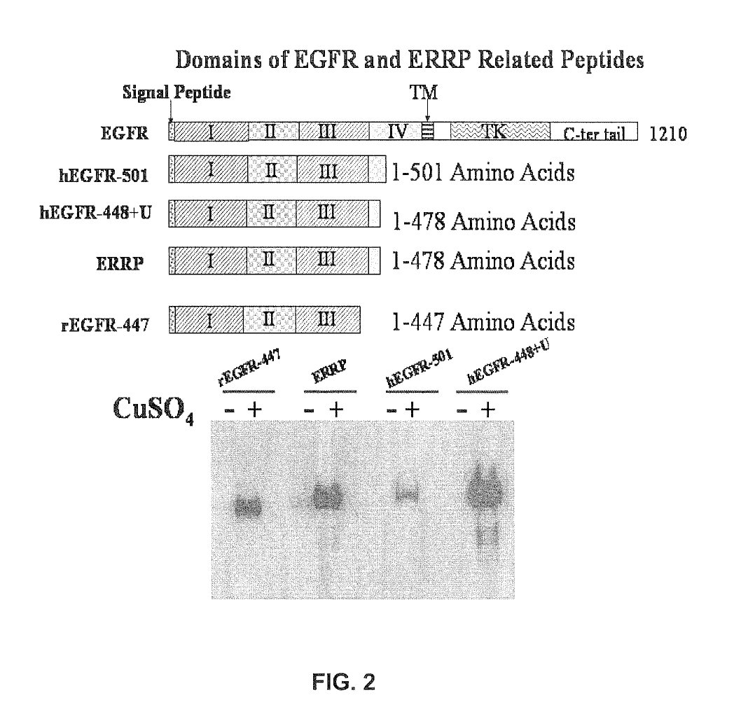

With reference to FIG. 2, a schematic representation of different peptides of EGFRs and their expression is shown. A schematic representation of full-length EGFR is also provided for comparison. The following experiments and results are obtained using the expression constructs and resultant peptides.

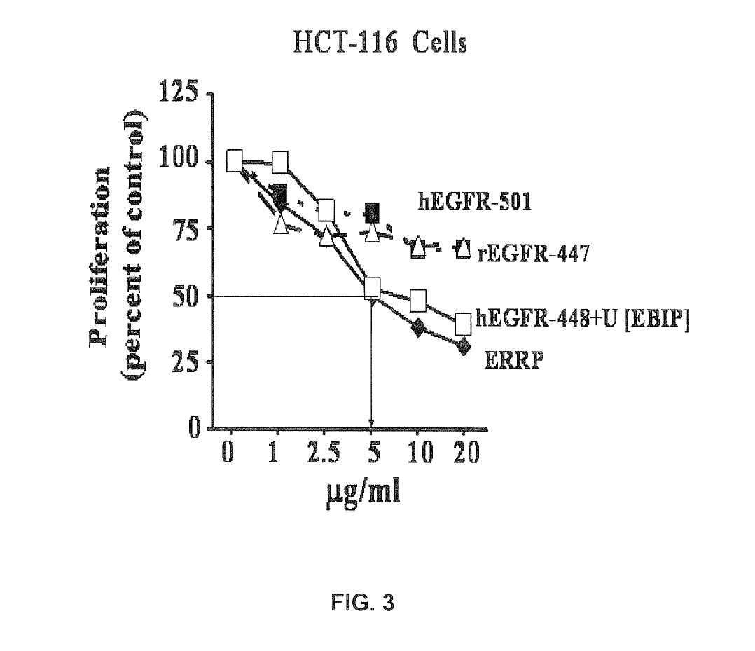

hEGFR containing “U” (hEGFR448+U; also referred to as EBIP) inhibit the growth of colon cancer HCT-116 cells in a dose dependent manner. However, peptides lacking ‘U” region (hEGFR-501 & rEGFR1-447) are only marginally effective in inhibiting growth, as illustrated in FIG. 3.

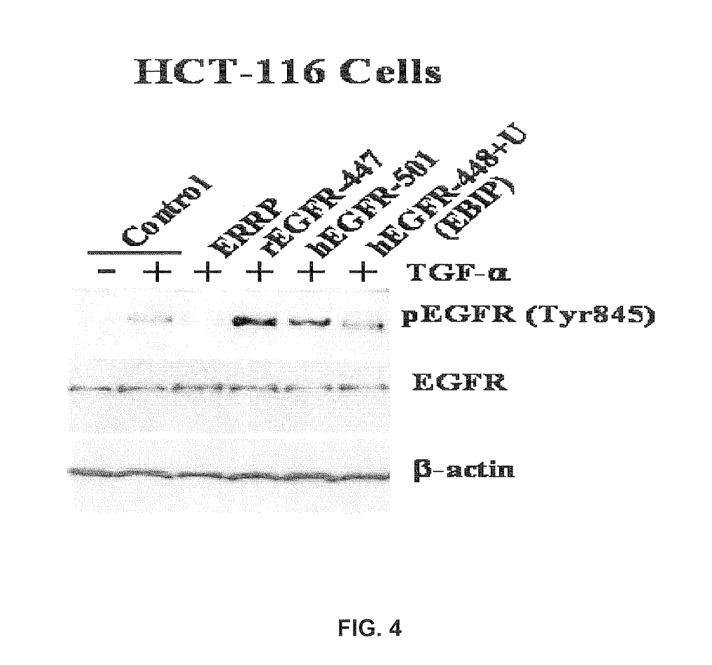

Accordingly, in various embodiments the “U” region in elicits the growth inhibitory properties of ERRP and human EGFR-448+U (EBIP). ERRP and EBIP (hEGFR1-448+U) may also attenuate TGF-α-induced activation of EGFR as determined by examining the levels of the tyrosine phosphorylated form of EGFR, as illustrated in FIG. 4. Exposure to 10 nM TGF-α for 10 min markedly stimulates tyrosine phosphorylation of the amino acid 845 of EGFR and preincubation of the cells with either ERRP or EBIP (hEGFR1-448+U) (10 μg/ml) for 30 min. greatly abrogates this stimulation (FIG. 4). In contrast, both rEGFR-447 and hEGFR-501 do not attenuate TGF-α-induced stimulation of tyrosine phosphorylation of EGFR (FIG. 4). In the short-term, the levels of total EGFR (unphosphorylated) remain unaffected by this treatment (FIG. 4).

The c-Src tyrosine kinase is involved in phosphorylation of the EGFR on Tyr-845, which is associated with modulation of receptor function. Tyrosine kinase activity of Src is regulated by tyrosine phosphorylation at two sites with opposing effects. Phosphorylation of Tyr-416 in the activation loop of the kinase domain upregulates the enzyme, while phosphorylation of Tyr-527 in the carboxy-terminal tail renders the enzyme less active.

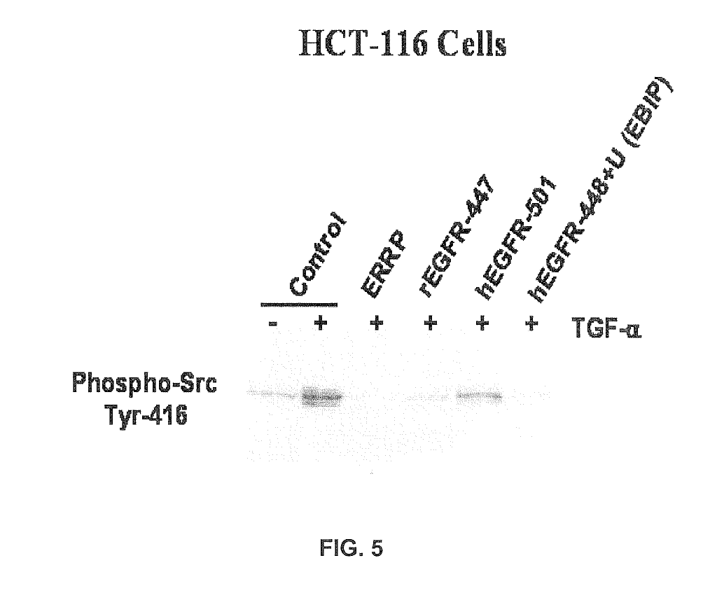

Referring now to FIG. 5, TGF-α markedly stimulates phosphorylation of Tyr416 of c-Src. This stimulation is abrogated by prior exposure to recombinant ERRP or hEGFR-448+U. On the other hand, less attenuation of TGF-α-induced Src activation is observed following prior exposure to rEGFR-447 and hEGFR-501.

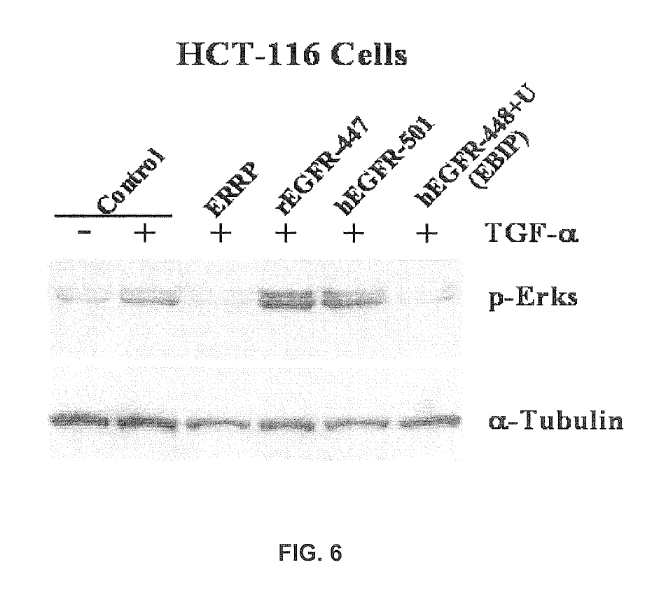

As observed for EGFR and Src phosphorylation, TGF-α markedly stimulates phosphorylation of Erks (extracellular signal-regulated kinases). This stimulation is greatly attenuated by prior exposure to ERRP or hEGFR+U, but not by prior exposure to rEGFR-447 or hEGFR-448 (EBIP), as illustrated in FIG. 6.

Accordingly, the “U” region is important in eliciting the growth inhibitory properties of human and rat EGFR ectodomains.

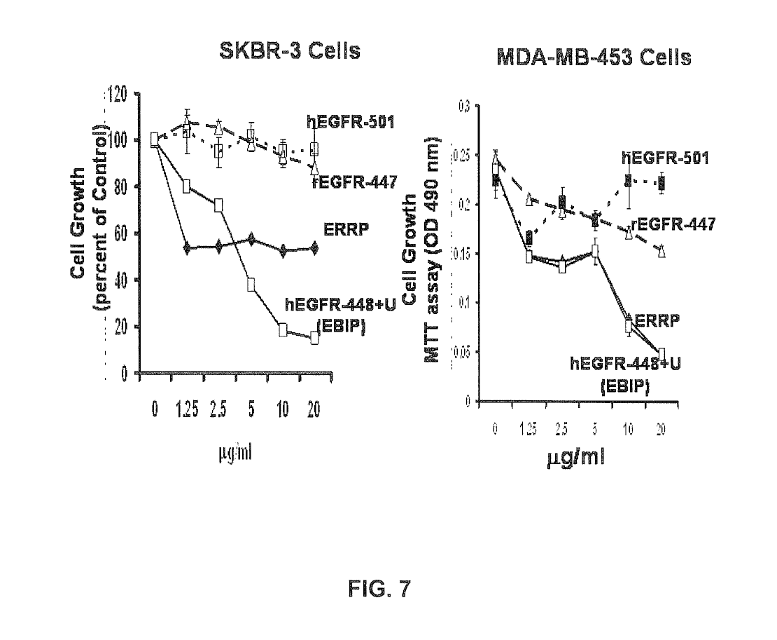

ERRP, a pan-erbB inhibitor, not only inhibits the growth of EGFR expressing cancer cells but also the cells that overexpress HER-2/ErbB-2. In various embodiments, different modified ectodomains of EGFR are effective in inhibiting the growth of HER-2 as well as HER-3 expressing cells. To demonstrate this inhibition, breast cancer SKBR-3 cells that express high levels of HER-2 and MDA-MB-453 cells that express high levels of HER-2 and HER-3 may be incubated in the absence (controls) or presence of increasing concentrations of ERRP, rEGFR-447, hEGFR-501 or hEGFR-448+U (EBIP) for 48 h. As shown in FIG. 7, whereas neither rEGFR-447 nor hEGFR-501 caused any apparent change in the growth of SKBR-3 cells, ERRP and hEGFR-447+U markedly inhibit the growth in a dose-dependent manner.

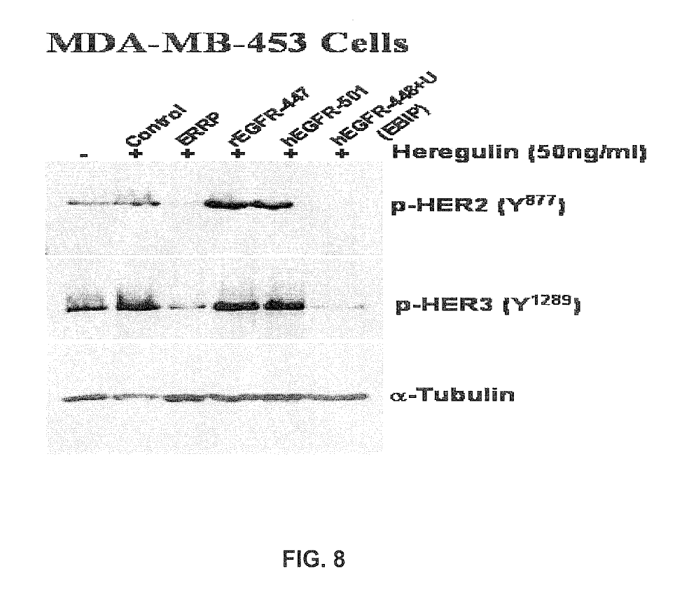

The breast cancer MDA-MB-453 cells contain high levels of HER-2 and HER-3. One of the ligands of HER-3 is heregulin. Binding of heregulin HER-3 leads to activation and subsequent heterodimerization of HER-3 with one or more EGFR family members. EBIP is also effective in abrogating heregulin-induced phosphorylation of HER-2 and HER-3 in MDA-MB-453 cells. Indeed, as shown in FIG. 8, exposure of serum-starved MDA-MB-453 cells to heregulin for 7 min stimulates tyrosine phosphorylation of HER-2 and HER-3, compared to the controls, while preincubation of the cells with EBIP abrogates this stimulation. ERRP is found to be about as equally effective as EBIP in abrogating stimulation. On the other hand, neither rEGFR-447 nor hEGFR-501 is effective in attenuating heregulin-induced stimulation of tyrosine phosphorylation of HER-2 or HER-3 in MDA-MB-453 cells, as shown in FIG. 8.

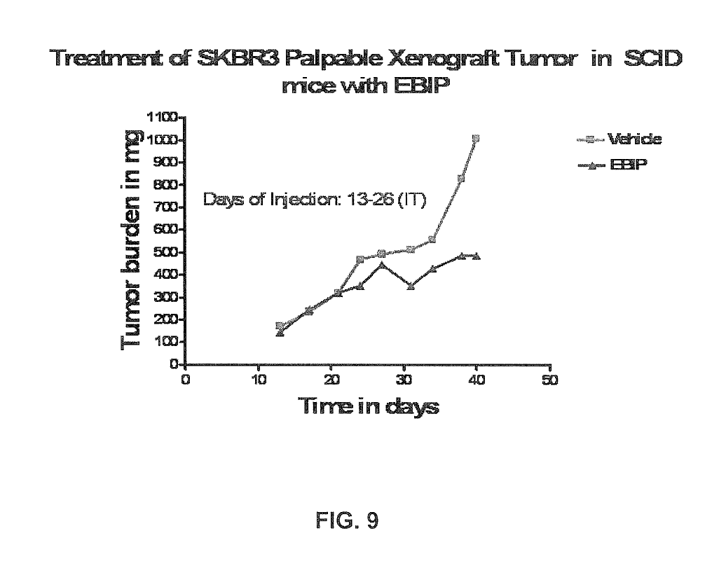

EBIP inhibits growth of breast cancer SKBR cell xenografts in SCID mice. SKBR cells express high levels of HER-2 but contain little no HER-1/EGFR or HER-3. This inhibition may be seen in tests essentially the same as described for colon cancer HCT-116 xenografts in SCID mice in Marciniak et al., Epidermal growth factor receptor related protein (ERRP): a potential therapeutic agent for colorectal cancer, Gastroenterology 124: 1337-1347, (2003), except that each mouse is injected (intratumoral; i.t.) with either ERRP (50 μg in 0.1 ml HEPES buffer, pH 7.7) or an equivalent volume of HEPES (controls), once a day from day 13 to day 26 of tumor implantation. The animals are sacrificed 40 days after tumor implantation; at which time the tumor weight in the control group is a little over 1000 mg, as seen in FIG. 9. With further reference to FIG. 9, the tumor growth rate is noticeably decreased in animals injected with ERRP compared to those treated with vehicle (controls). At the end of the 40 day experimental period, intratumoral (i.t.) injection of ERRP causes a little over 50% inhibition of tumor growth, when compared with the controls (FIG. 9).

Accordingly, that incorporation of the “U” epitope at the carboxy-terminus of the ectodomain of the human EGFR (containing amino acids 1-448) enhances the growth inhibitory properties of this molecule. The hEGFR-448+U (EBIP) is useful as an anti-cancer agent for tumors whose growth is dependent on signaling induced by EGFR and/or its family members.

EBIP has 94% identity with human EGFR and is unlikely to provoke adverse immune response since the EGFR component is of human origin and the “U” region harbors an antigenic epitope that is present in human proteins, as evidenced by the cross reactivity with antibodies generated against the “U” region epitope, described by Marciniak et al., Epidermal growth factor receptor related protein (ERRP): a potential therapeutic agent for colorectal cancer, Gastroenterlogy 124: 1337-1347 (2003); Feng et al., Expression of ERRP in Normal and Neoplastic Pancreata And its Relationship to Clinicopathological Parameters in Pancreatic Adenocarcinoma, Pancreas 25: 342-349, (2002); and Jaszewski et al., Expression of Epidermal Growth Factor-Receptor Related Protein (ERRP) in Human Colorectal Carcinogenesis, Cancer Lett. 213: 249-225 (2004).

The present technology provides compositions and methods that include a protein comprising a first portion that binds a ligand of an EGFR or EGFR family member and a second portion that includes the “U” region epitope of ERRP. Embodiments also include or use a portion of the ectodomain of EGFR fused to the “U” region epitope of ERRP and nucleic acids encoding a portion of the ectodomain of EGFR fused to the “U” region epitope of ERRP. For example, a portion of the EGFR ectodomain may bind a ligand of an EGFR.

Referring again to FIG. 2, the present proteins may include domains I, II, and III of EGFR, ERRP, and related peptides plus the “U” region. Nucleic acids include those encoding domains I, II, and III of EGFR and ERRP related peptides plus the “U” region. These include proteins and/or nucleic acids encoding the proteins hEGFR448+U (EBIP) (SEQ ID NO: 14) and rat ERRP that includes amino acids 1-478 includes the “U” region (ERRP) (SEQ ID NO: 16). “U” region is included in 1-178 amino acids. The present technology further includes homologues and derivatives of these proteins as well as homologues and derivatives of nucleic acids encoding these proteins. Also included are purified and isolated forms, recombinant forms, and synthetic forms of these proteins and nucleic acids. Thus, embodiments of the present technology that include SEQ ID NOS: 14 and 16 also include homologues and derivatives thereof, as well as purified, isolated, recombinant, and synthetic &onus thereof.

In some embodiments, proteins of the present technology include: an isolated protein comprising a portion of the EGFR and the “U” region epitope of ERRP, wherein the portion of the EGFR binds a ligand of EGFR; an isolated protein comprising a portion of the ERRP and the “U” region epitope of ERRP, wherein the portion of the ERRP binds a ligand of ERRP; an isolated protein comprising the extracellular domain (ectodomain) of human EGFR and the “U” region epitope of ERRP, or the extracellular domain (ectodomain) of a mammalian EGFR homologue and the “U” region epitope of ERRP; an isolated protein comprising domains I, II, III of EGFR and the “U” region epitope of ERRP; an isolated protein comprising SEQ ID NO: 14 or SEQ ID NO: 16; an isolated protein consisting of SEQ ID NO: 14 or SEQ ID NO: 16; an isolated variant of a protein comprising the amino acid sequence shown in SEQ ID NO: 14, wherein the variant comprises an amino acid sequence that is at least 80%, 85%, 90%, 95%, or 98% homologous or identical to SEQ ID NO: 14; and an isolated variant of a protein comprising the amino acid sequence shown in SEQ ID NO: 16, wherein the variant comprises an amino acid sequence that is at least 80%, 85%, 90%, 95%, or 98% homologous or identical to SEQ ID NO: 16.

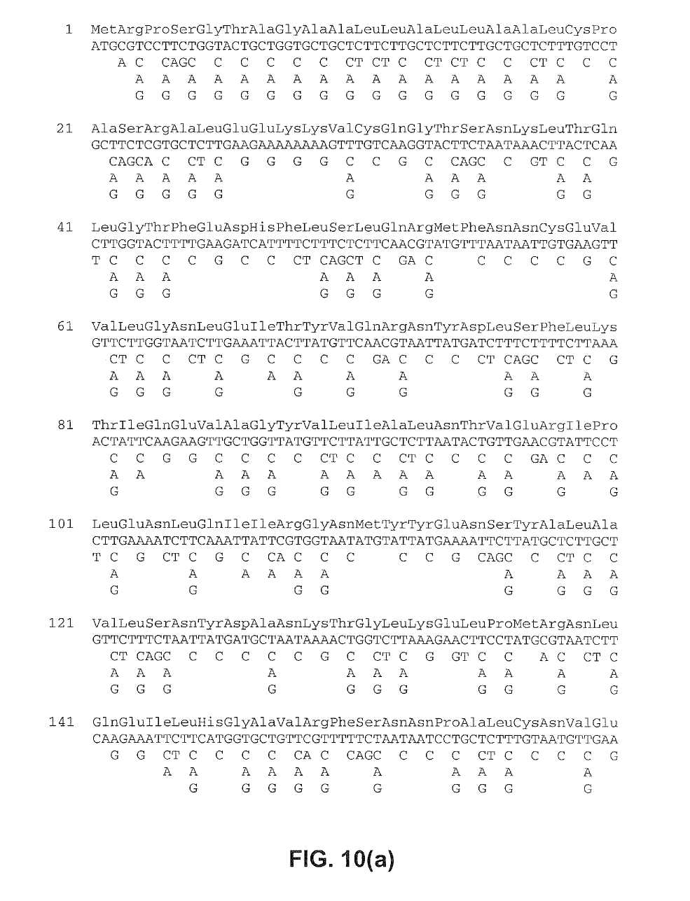

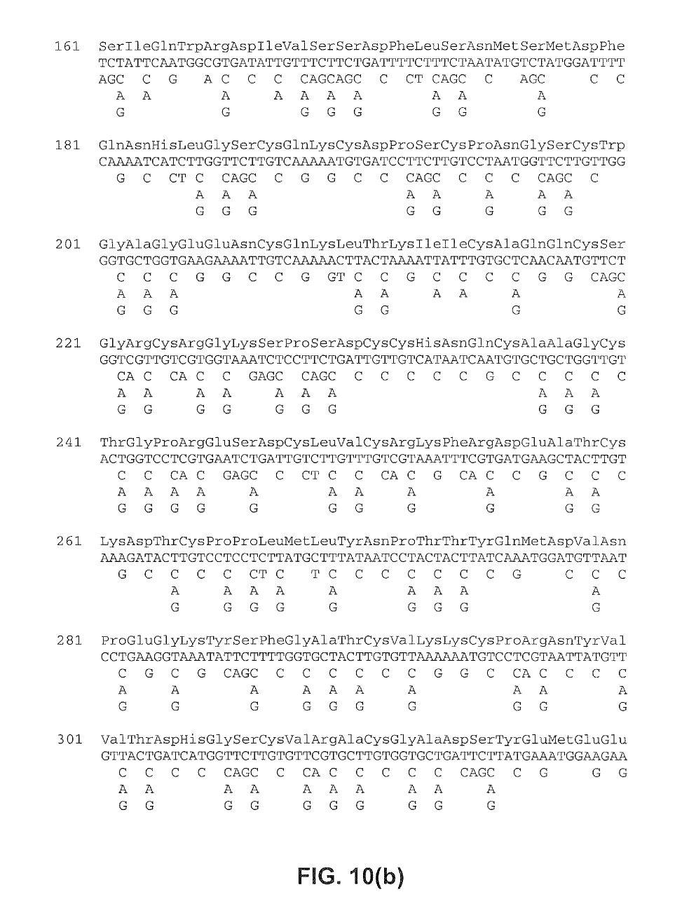

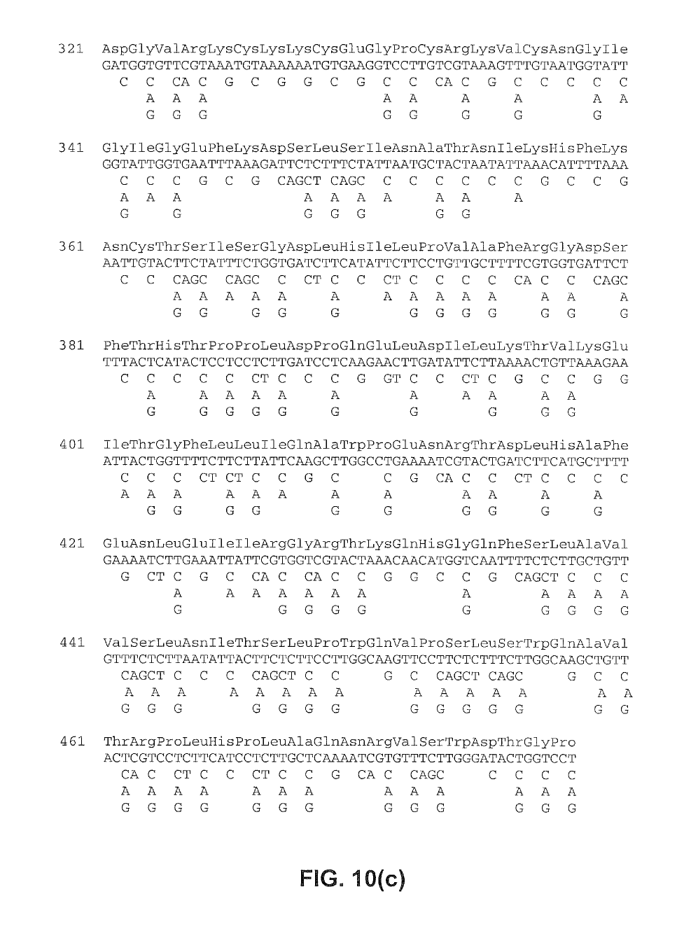

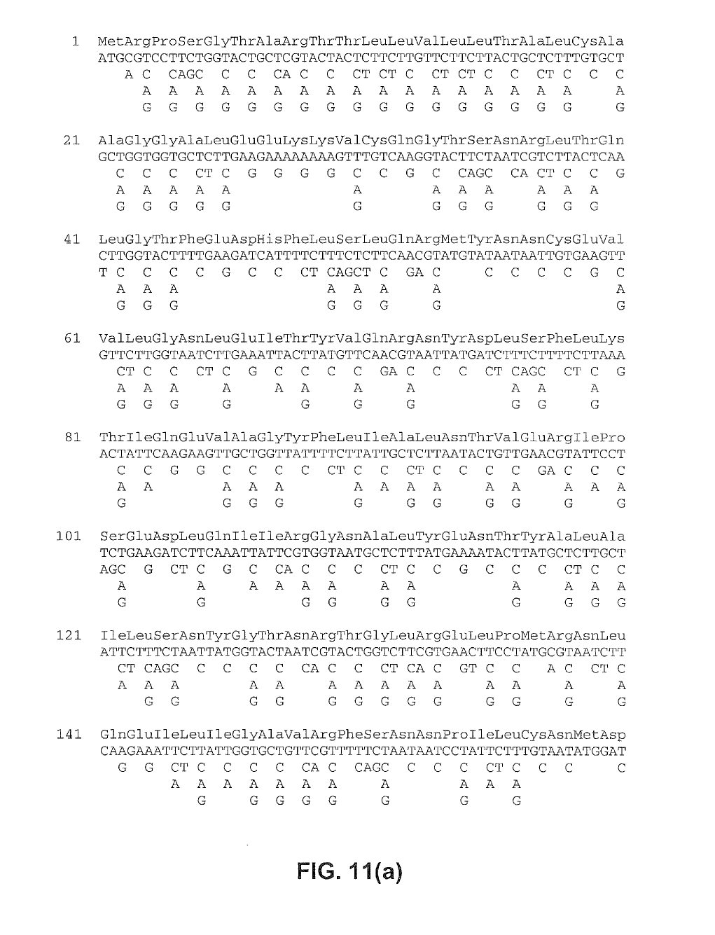

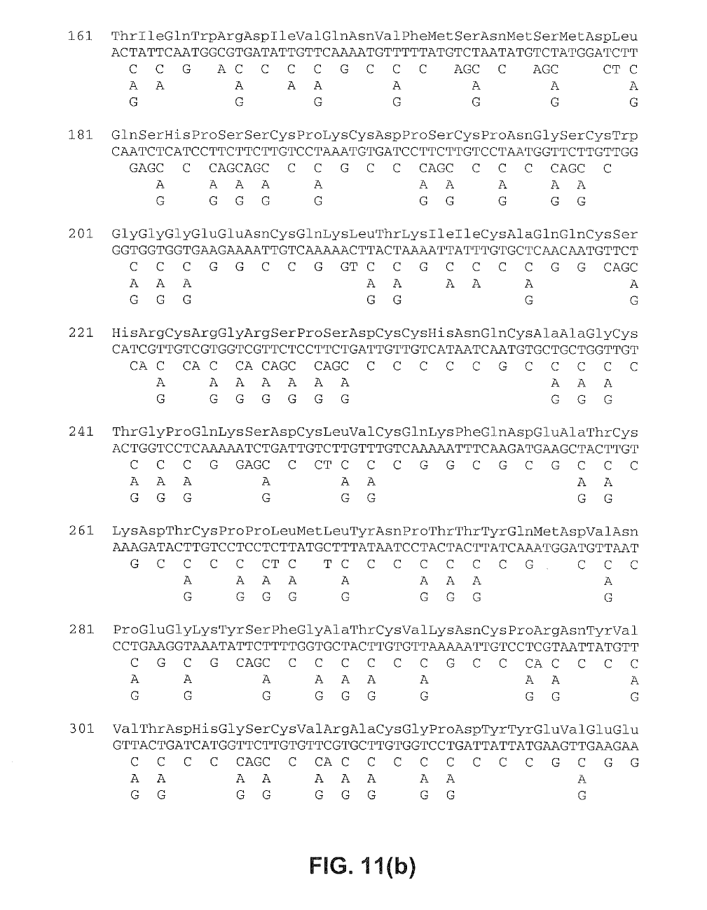

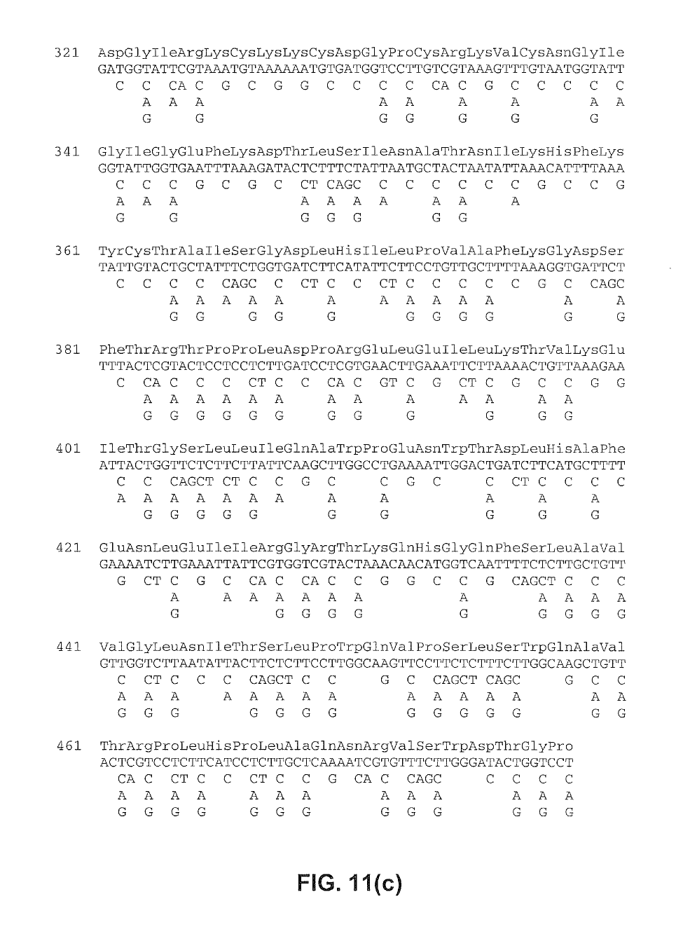

In some embodiments, the present technology includes nucleic acids encoding for each of the preceding proteins, such as an isolated DNA that encodes one of the aforementioned proteins. For example, the present technology includes an isolated DNA that encodes a protein having the amino acid sequence of SEQ ID NO: 14 or SEQ ID NO: 16. Examples of nucleic acids that encode the amino acid sequences of SEQ ID NO: 14 and SEQ ID NO: 16 are shown in FIGS. 10(a)-(c) and FIGS. 11(a)-(d), respectively. For example, reverse translated DNA sequences of the amino acid sequences of SEQ ID NO: 14 and SEQ ID NO: 16 include the DNA sequences shown in SEQ ID NO: 17 and SEQ ID NO: 18, respectively. The skilled artisan appreciates that the degeneracy of the genetic code affords variations in nucleic acid sequence that encode the same amino acid sequence. The skilled artisan also appreciates that expression of proteins in some organisms may include differences in codon usage and may also include the use of nonstandard codons. Such variations in nucleic acid sequence are part of the present technology.

The present technology also provides isolated polypeptides and nucleic acids comprising the sequences as set forth in SEQ ID NOS: 14 and 16. As used herein, “isolated,” when referring to a molecule or composition, such as, e.g., an isolated infected cell comprising a nucleic acid sequence, means that the molecule or composition (including, e.g., a cell) is separated from at least one other compound, such as a protein, DNA, RNA, or other contaminants with which it is associated in vivo or in its naturally occurring state. Thus, a nucleic acid or polypeptide or peptide sequence is considered isolated when it has been isolated from any other component with which it is naturally associated. An isolated composition can, however, also be substantially pure. An isolated composition can be in a homogeneous state. It can be dry or an aqueous or organic solution. Purity and homogeneity can be determined using any analytical chemistry technique, for example.

As used herein, the term “nucleic acid” or “nucleic acid sequence” refers to a deoxyribonucleotide or ribonucleotide oligonucleotide, including single- or double-stranded, or coding or non-coding (e.g., “antisense”) forms. The term encompasses nucleic acids, i.e., oligonucleotides, containing known analogues of natural nucleotides. The term also encompasses nucleic-acid-like structures with synthetic backbones, see e.g., Oligonucleotides and Analogues, a Practical Approach, ed. F. Eckstein, Oxford Univ. Press (1991); Antisense Strategies, Annals of the N.Y. Academy of Sciences, Vol 600, Eds. Baserga et al. (NYAS 1992); Milligan (1993) J. Med. Chem. 36:1923-1937; Antisense Research and Applications (1993, CRC Press), WO 97/03211; WO 96/39154; Mata (1997) Toxicol. Appl. Pharmacol. 144:189-197; Strauss-Soukup (1997) Biochemistry 36:8692-8698; and Samstag (1996) Antisense Nucleic Acid Drug Dev 6:153-156. As used herein, the “sequence” of a nucleic acid or gene refers to the order of nucleotides in the polynucleotide, including either or both strands (sense and antisense) of a double-stranded DNA molecule, e.g., the sequence of both the coding strand and its complement, or of a single-stranded nucleic acid molecule (sense or anti sense). For example, in alternative embodiments, promoters drive the transcription of sense and/or antisense polynucleotide sequences of the present technology.

The terms “polypeptide,” “protein,” and “peptide” include compositions of the present technology that also include “analogs,” or “conservative variants” and “mimetics” (“peptidomimetics”) with structures and activity that substantially correspond to the exemplary sequences. Thus, the terms “conservative variant” or “analog” or “mimetic” also refer to a polypeptide or peptide which has a modified amino acid sequence, such that the change(s) do not substantially alter the polypeptide's (the conservative variant's) structure and/or activity (e.g., immunogenicity, ability to bind to human antibodies, etc.), as defined herein. These include conservatively modified variations of an amino acid sequence, i.e., amino acid substitutions, additions, or deletions of those residues that are not critical for protein activity, or substitution of amino acids with residues having similar properties (e.g., acidic, basic, positively or negatively charged, polar or non-polar, etc.) such that substitution(s) of even critical amino acids do not substantially alter structure and/or activity.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gin; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); and 6) Phenylalanine (F), Tyrosine (Y), Tryptophan (W); (see also, e.g., Creighton (1984) Proteins, W.H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions, or additions that alter, add, or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides of the present technology (e.g., ability to bind, or “capture,” human antibodies in an ELISA). The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions; as long as such substitutions also do not substantially alter the mimetic's structure and/or activity. As with polypeptides of the present technology which are conservative variants, routine experimentation will determine whether a mimetic is within the scope of the technology; i.e., that its structure and/or function is not substantially altered.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, N.Y.). A polypeptide can also be characterized as a mimetic by containing all or some non-natural residues in place of naturally occurring amino acid residues; non-natural residues are well described in the scientific and patent literature.

The present technology comprises nucleic acids comprising sequences encoding the proteins of SEQ ID NOS: 14 and 16, or comprising nucleic acids encoding such proteins operably linked to a transcriptional regulatory sequence. As used herein, the term “operably linked,” refers to a functional relationship between two or more nucleic acid (e.g., DNA) segments. Typically, it refers to the functional relationship of a transcriptional regulatory sequence to a transcribed sequence. For example, a promoter (defined below) is operably linked to a coding sequence, such as a nucleic acid of the present technology, if it stimulates or modulates the transcription of the coding sequence in an appropriate host cell or other expression system. Generally, promoter transcriptional regulatory sequences that are operably linked to a transcribed sequence are physically contiguous to the transcribed sequence; i.e., they are cis-acting. However, some transcriptional regulatory sequences, such as enhancers, need not be physically contiguous or located in close proximity to the coding sequences whose transcription they enhance; i.e., trans-acting. For example, in one embodiment, a promoter is operably linked to an ORF-containing nucleic acid sequence of the present technology.

As used herein, the term “promoter” includes all sequences capable of driving transcription of a coding sequence in an expression system. Thus, promoters used in the present constructs include cis-acting transcriptional control elements and regulatory sequences that are involved in regulating or modulating the timing and/or rate of transcription of a nucleic acid of the present technology. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

The present technology provides expression cassettes comprising nucleic acids encoding the proteins of SEQ ID NOS: 14 and 16. The term “expression vector” refers to any recombinant expression system for the purpose of expressing a nucleic acid sequence of the present technology in vitro or in vivo, constitutively or inducibly, in any cell, including prokaryotic, yeast, fungal, plant, insect, or mammalian cell. The term includes linear or circular expression systems. The term includes expression systems that remain episomal or that integrate into the host cell genome. The expression systems can have the ability to self-replicate or not; i.e., drive only transient expression in a cell. The term includes recombinant “expression cassettes” which contain only the minimum elements needed for transcription of the recombinant nucleic acid.

Alignment analysis of nucleic acid sequences encoding SEQ ID NOS: 14 and 16 includes the following aspects. The nucleic acid and polypeptide sequences of the present technology include genes and gene products identified and characterized by sequence identify analysis (i.e., by homology) using the exemplary nucleic acid and protein sequences of SEQ ID NOS: 14 and 16. In various aspects of the present technology, nucleic acids and polypeptides within the scope of the present technology include those having 98%, 95%, 90%, 85%, or 80% sequence identity (homology) to the sequences as set forth in SEQ ID NOS: 14 and 16.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used unless alternative parameters are designated herein. The sequence comparison algorithm then calculates the percent sequence identity for the test sequence(s) relative to the reference sequence, based on the designated or default program parameters. A “comparison window,” as used herein, includes reference to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (CLUSTAL, GAP, BESTFIT, FASTA, and TFASTA in the Wisconsin Genetics Software Package, Genetics Computer Group, 575 Science Dr., Madison, Wis.), or by manual alignment and visual inspection.

In some aspects of the present technology (in the methods of the present technology, and, to determine if a sequence is within the scope of the present technology), a CLUSTAL algorithm is used, e.g., the CLUSTAL W program, see, e.g., Thompson (1994) Nuc. Acids Res. 22:4673-4680; Higgins (1996) Methods Enzymol 266:383-402. Variations can also be used, such as CLUSTAL X, see Jeanmougin (1998) Trends Biochem Sci 23:403-405; Thompson (1997) Nucleic Acids Res 25:4876-4882. In one aspect, the CLUSTAL W program described by Thompson (1994) supra, is used with the following parameters: K tuple (word) size: 1, window size: 5, scoring method: percentage, number of top diagonals: 5, gap penalty: 3, to determine whether a nucleic acid has sufficient sequence identity to an exemplary sequence to be with the scope of the present technology.

In other aspects, the algorithm PILEUP can be used in the present methods and can be used to determine whether a nucleic acid has sufficient sequence identity to be with the scope of the present technology. This program creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). Using PILEUP, a reference sequence is compared to another sequence to determine the percent sequence identity relationship (i.e., that the second sequence is substantially identical and within the scope of the present technology) using the following parameters: default gap weight (3.00), default gap length weight (0.10), and weighted end gaps.

In some embodiments, PILEUP obtained from the GCG sequence analysis software package, e.g., version 7.0 (Devereaux (1984) Nuc. Acids Res. 12:387-395), using the parameters described therein, is used in the methods and to identify nucleic acids within the scope of the present technology. In another aspect, a BLAST algorithm is used (in the methods, e.g., to determine percent sequence identity (i.e., substantial similarity or identity) and whether a nucleic acid is within the scope of the present technology), see, e.g., Altschul (1990) J. Mol. Biol. 215:403-410. Software for performing BLAST analyses is publicly available through the National Center for Biotechnology Information, NIH. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues, always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment.

In some embodiments, to determine if a nucleic acid sequence is within the scope of the present technology, the BLASTN program (for nucleotide sequences) is used incorporating as defaults a wordlength (W) of 11, an expectation (E) of 10, M=5, N=4, and a comparison of both strands. For amino acid sequences, the BLASTP program uses as default parameters a wordlength (W) of 3, an expectation (E) of 10, and the BLOSUM62 scoring matrix; see, e.g., Henikoff (1989) Proc. Natl. Acad. Sci. USA 89:10915.

Hybridization for identifying nucleic acids of the present technology can include the following aspects. Nucleic acids include isolated or recombinant nucleic acids that specifically hybridize under stringent hybridization conditions to an exemplary nucleic acid of the present technology (including a sequence encoding an exemplary polypeptide) as set forth in SEQ ID NOS: 14 and 16. Stringent conditions are sequence-dependent and will be different in different circumstances. Longer sequences hybridize specifically at higher temperatures. An extensive guide to the hybridization of nucleic acids is found in, e.g., Tijssen, P (1993) Hybridization with Nucleic Acid Probes, Part I: Theory and Nucleic Acid Preparation; Elsevier, Amsterdam, The Netherlands, pp. 1-17. Generally, stringent conditions are selected to be about 5° C. to about 10° C. lower than thermal melting point (Tₘ) for the specific sequence at a defined ionic strength and pH. The Tₘ is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents, such as formamide.

For selective or specific hybridization, a positive signal (e.g., identification of a nucleic acid of the present technology) is about 10 times background hybridization. “Stringent” hybridization conditions that are used to identify substantially identical nucleic acids within the scope of the present technology include hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary “moderately stringent hybridization conditions” include hybridization in a buffer of 40% formamide, 1 M NaCl, and 1% SDS at 37° C., and a wash in 1×SSC at 45° C. Those of ordinary skill will readily recognize that alternative but comparable hybridization and wash conditions can be utilized to provide conditions of similar stringency.

Nucleic acids that do not hybridize to each other under stringent hybridization conditions are still substantially identical if the polypeptides which they encode are substantially identical. This may occur, for example, when a copy of a nucleic acid is created using the maximum codon degeneracy permitted by the genetic code, as discussed herein (see discussion on “conservative substitutions”). However, the selection of a hybridization format is not critical—it is the stringency of the wash conditions that set forth the conditions that determine whether a nucleic acid is within the scope of the present technology. Wash conditions used to identify nucleic acids within the scope of the present technology include: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook and Tijssen as disclosed herein for descriptions of SSC buffer and equivalent conditions, and see Current Protocols in Molecular Biology, F. Ausubel et al., ed. Greene Publishing and Wiley-Interscience, New York (1987).

The nucleic acid and polypeptide sequences of the present technology and other nucleic acids used to practice this technology, whether RNA, cDNA, genomic DNA, vectors, viruses or hybrids thereof, may be isolated from a variety of sources, genetically engineered, amplified, and/or expressed recombinantly. Any recombinant expression system can be used, including, in addition to bacterial cells, e.g., mammalian, yeast, insect, or plant cell expression systems.

Alternatively, these nucleic acids and polypeptides can be synthesized in vitro by well-known chemical synthesis techniques, as described in, e.g., Caruthers (1982) Cold. Spring Harbor Symp. Quant Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, generating mutations in sequences, sub-cloning, labeling probes, sequencing, hybridization and the like are well described in the scientific and patent literature, see, e.g., Sambrook, ed., Molecular Cloning; A Laboratory Manual (2nd Ed.), Vols. 1-3, Cold Spring Harbor Laboratory, (1989); Current Protocols in Molecular Biology, Ausubel, Ed. John Wiley & Sons, Inc., New York (1997); Laboratory Techniques in Biochemistry and Molecular Biology Hybridization with Nucleic Acid Probes, Part I. Theory and Nucleic Acid Preparation, Tijssen, ed. Elsevier, N.Y. (1993).

Polypeptides and peptides of the present technology can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431 A Peptide Synthesizer (Perkin Elmer) in accordance with the instructions provided by the manufacturer.

The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Peptides and peptide mimetics can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the present technology can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

Peptides and polypeptides can also be synthesized and expressed as fusion proteins with one or more additional domains linked thereto for producing a more immunogenic peptide, to more readily isolate a recombinantly synthesized peptide, to identify and isolate antibodies and antibody-expressing B cells, and the like. Detection and purification facilitating domains include metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between the purification domain and GCA-associated peptide or polypeptide can be useful to facilitate purification. For example, an expression vector can include an epitope-encoding nucleic acid sequence linked to six histidine residues followed by a thioredoxin and an enterokinase cleavage site; e.g., Williams (1995) Biochemistry 34:1787-1797; Dobeli (1998) Protein Expr. Purif. 12:404414. The histidine residues can facilitate detection and purification while the enterokinase cleavage site provides a means for purifying the epitope from the remainder of the fusion protein. Technology pertaining to vectors encoding fusion proteins and application of fusion proteins are well described in the scientific and patent literature; see e.g., Kroll (1993) DNA Cell. Biol., 12:441-53.

The present technology also provides antibodies that specifically bind to the polypeptides of SEQ ID NOS: 14 and 16. These antibodies can be useful in the screening and/or purification methods. The polypeptides or peptide can be conjugated to another molecule or can be administered with an adjuvant. The coding sequence can be part of an expression cassette or vector capable of expressing the immunogen in vivo; e.g., Katsumi (1994) Hum. Gene Ther. 5:1335-9. Methods of producing polyclonal and monoclonal antibodies are known to those of skill in the art and described in the scientific and patent literature; see, e.g., Coligan, Current Protocols in Immunology, Wiley/Greene, N.Y. (1991); Stites (eds.) Basic and Clinical Immunology (7th ed.) Lange Medical Publications, Los Altos, Calif.; Goding, Monoclonal Antibodies: Principles and Practice (2d ed.) Academic Press, New York, N.Y. (1986); and Harlow (1988) Antibodies, A Laboratory Manual, Cold Spring Harbor Publications, New York.

Antibodies also can be generated in vitro using recombinant antibody binding site expressing phage display libraries in addition to the traditional in vivo methods using animals. See, e.g., Huse (1989) Science 246:1275; Ward (1989) Nature 341:544; Hoogenboom (1997) Trends Biotechnol. 15:62-70; Katz (1997) Annu. Rev. Biophys. Biomol. Struct. 26:27-45. Human antibodies can be generated in mice engineered to produce only human antibodies, as described by, e.g., U.S. Pat. Nos. 5,877,397, Lonberg et al., issued Mar. 2, 1999; 5,874,299, Lonberg et al., issued Feb. 23, 1999; 5,789,650, Lonberg et al., issued Aug. 4, 1998; and 5,939,598, Kucherlapati et al., issued Aug. 17, 1999. B-cells from these mice can be immortalized using standard techniques (e.g., by fusing with an immortalizing cell line such as a myeloma or by manipulating such B-cells by other techniques to perpetuate a cell line) to produce a monoclonal human antibody-producing cell. See, e.g., U.S. Pat. No. 5,916,771, Hori et al., issued Jun. 29, 1999 and U.S. Pat. No. 5,985,615, Jakobovits et al., issued Nov. 16, 1999.

Proteins and nucleic acids of the present technology are useful in attenuating signaling via the EFGR and EGFR family members. This property may be used in treatments and therapies where inhibition of cellular growth is desirable where the target cells express one or more of the EGFR and EGFR family members. For example, the modified ectodomain of human EGFR that contains the 30-amino acid “U” epitope of ERRP (referred to as hEGFR-448+U or EBIP, ErbB Inhibitory Protein) may be used as an anticancer agent for tumors whose growth is dependent EGFR and/or its family members, specifically HER-2. Since hEGFR-448+U/EBIP is of human origin, no adverse toxicity or immunogenicity would be expected.

Aspects of the present technology further include methods and compositions that employ a combination of an EGFR with a modified ectodomain and an inhibitor of a non-receptor tyrosine kinase. Many solid tumors, including breast cancer, show increased activation of several growth factor receptors, specifically epidermal growth factor receptor (EGFR) and its family members as well as c-Src, a non-receptor tyrosine kinase that promotes proliferation, inhibits apoptosis, and induces metastasis. Inhibition of both c-Src and EGFRs can be an effective therapeutic strategy for triple-negative breast cancer.

In some embodiments, the c-Src-specific inhibitor dasatinib (BMS-354825; Bristol-Myers Squibb) is employed in conjunction with an ErbB-inhibitory protein (EBIP), a potential pan-ErbB inhibitor, to treat breast cancer. As described herein, EBIP is composed of amino acids 1 to 448 of the ectodomain of human EGFR to which the 30-amino acid epitope (the “U” region) of rat EGFR-related protein is fused at the COOH-terminal end. The combination of dasatinib and EBIP is highly effective in inhibiting the growth of four different breast cancer cells (MDA-MB-468, SKBr-3, MDA-MB-453, and MDA-MB-231) that express different levels of EGFRs. In EGFR-overexpressing MDA-MB-468 cells, the combination, but not monotherapy, markedly stimulated apoptosis mediated by caspase-9 and caspase-8 and attenuated activation of EGFR and Src as well as tyrosine kinase activity. EBIP also inhibited heregulin-induced activation of HER-2 and HER-3 in MDA-MB-453 breast cancer cells. The combination therapy was highly effective in suppressing tumor growth (about 90% inhibition) in MDA-MB-468-derived xenografts in severe combined immunodeficient mice. The latter could be attributed to induction of apoptosis. Combining a non-receptor tyrosine kinase inhibitor (e.g., dasatinib) with an EGFR with a modified ectodomain (e.g., EBIP) can be an effective therapeutic strategy for breast cancer by targeting signaling by both EGFRs and Src.

Several targeted therapies for epidermal growth factor receptor (EGFR) and its family members have been developed for treatment of many malignancies, including breast cancers. Although trastuzumab, monoclonal antibodies to HER-2, is being used for treatment of HER-2-overexpressing breast cancer, it is not an effective therapy for triple-negative breast cancer. The fact that the extracellular or ectodomain of EGFR is essential for ligand binding and subsequent homodimerization/heterodimerization of the receptor raises the possibility that this domain of EGFR could be used to inhibit EGFR functions and could, therefore, be developed into an anticancer agent. Indeed, EGFR-related protein (ERRP), a 53- to 55-kDa protein, which we isolated from the rat gastroduodenal mucosa, targets multiple members of the EGFR family and inhibits growth of several epithelial cancers, including the gastric mucosa, colon, and pancreas.

As disclosed herein, ERRP is composed of three of the four extracellular subdomains of EGFR and a 30-amino acid unique epitope (termed “U”) at the COOH terminus. Although the 30-amino acid U region of ERRP possesses no homology with any known protein, antibodies raised against this epitope strongly cross-react with proteins in the liver, pancreas, gastric mucosa, and colon of humans. This suggests that the U region harbors an antigenic epitope that is present in humans. The fact that ERRP was isolated from the rat that showed about 85% homology to the ectodomain of human EGFR (hEGFR) raised the possibility of inducing antigenic response in humans. This had prompted us to generate the expression plasmid of truncated hEGFR ectodomain containing the U region of ERRP, which is referred to as ErbB-inhibitory protein (EBIP).

The following experiments demonstrate the growth-inhibitory properties of EBIP in breast cancer cells that express varying levels of EGFR and its family members. Furthermore, the fact that c-Src may also be involved in the development and progression of breast cancer led us to study the effectiveness of the c-Src inhibitor dasatinib (BMS-354825), alone or together with EBIP, in inhibiting growth of breast cancer cells. Dasatinib (BMS-354825) was identified as a highly potent, ATP-competitive inhibitor of Src family kinases and Abl kinases, which is approved for imatinib-resistant chronic myelogenous leukemia and (Philadelphia chromosome-positive) acute lymphoblastic leukemia treatment. Dasatinib has been shown to exhibit antiproliferative activity in both hematologic and solid tumors and is currently in clinical trial for triple-negative breast cancer. Dasatinib has also been shown to inhibit processes of metastasis such as migration and invasion.

Cell lines and cell culture included the following aspects. Human breast cancers MDA-MB-468, SKBr-3, MDAMB-453, and MDA-MB-231 cells, obtained from the American Type Culture Collection, were used to investigate the mechanisms of growth inhibition by dasatinib and/or EBIP. All cell lines were maintained in DMEM, as described by Nautiyal J, Majumder P, Patel B B, Lee F Y, Majumdar A P, “Src inhibitor dasatinib inhibits growth of breast cancer cells by modulating EGFR signaling,” Cancer Lett. 2009.

Chemicals and sources included the following. DMEM, fetal bovine serum, and antibiotic/antimycotic were obtained from Life Technologies. Dasatinib was obtained partially from Bristol-Myers Squibb through Material Transfer Agreement and purchased from LC Laboratories. Protease inhibitor cocktail, MTT, and other chemicals were obtained from Sigma. Acridine orange and ethidium bromide were purchased from BD Biosciences. Acridine orange/ethidium bromide mixture was prepared according to the manufacturer's instruction. Anti-phospho-EGFR (Tyr845), phospho-EGFR (Tyr1173), phospho-HER-2 (Tyr877), phospho-HER-3 (Tyr1289), phospho-Akt (Ser473), phospho-extracellular signal-regulated kinase p44/42, c-Src, and phospho-Src (Tyr416) were purchased from Cell Signaling. Antibodies to β-actin were purchased from Chemicon International, Inc. Recombinant transforming growth factor-α (TGF-α) and heregulin were procured from Calbiochem. Antibodies to α-tubulin were purchased from Oncogene. Antibodies to poly(ADP-ribose) polymerase (PARP) and EGFR were obtained from Santa Cruz Biotechnology, Inc., and anti-V5 were purchased from Invitrogen. In Situ Cell Death Detection Kit, POD was obtained from Roche Diagnostics GmbH to do terminal deoxynucleotidyl transferase-mediated dUTP nick end labeling (TUNEL) assay.

Generation of EBIP expression constructs included the following aspects. The following expression constructs were generated.

Rat EGFR ectodomain (ERRP without U region; referred to as ERRP-447): Rat EGFR sequences corresponding to ERRP (amino acids 1-447) were PCR amplified using the following primers: (forward) 5′-ATGCGACCCTCAGGGACCGCGAG-3′ (SEQ ID NO: 1) and (reverse) 5′-CCGCTCGAGGATGTTATGTTCAGGCCGAC-3′ (SEQ ID NO: 2). The PCR product was cut with XhoI restriction enzymes and subcloned into EcoRV+XhoI cut pMT/His-V5B vector (Invitrogen) to obtain a recombinant plasmid for expression of V5-His-tagged rat EGFR ectodomain sequences.

hEGFR ectodomain (referred to as hEGFR-501): hEGFR sequences from amino acids 1 to 501 were PCR amplified using the following primers: (forward) 5′-CGCAAGCTTCGGGAGAGCCGGAGCGAGC-3′ (SEQ ID NO: 3) and (reverse) 5′-CCGCTCGAGGCCTTGCAGCTGTTTTCAC-3′ (SEQ ID NO: 4). The reason for selecting position 501 for truncation was that this truncated ectodomain of hEGFR was shown by Elleman et al. (Elleman T C, Domagala T, McKern N M, et al. “Identification of a determinant of epidermal growth factor receptor ligand-binding specificity using a truncated, high-affinity form of the ectodomain.” Biochemistry 2001; 40:8930-9) to bind EGFR ligands (e.g., EGF and TGF-α) with 13- to 14-fold higher affinity than the full-length EGFR ectodomain. The PCR product was cut with XhoI restriction enzyme and subcloned into EcoRV+XhoI cut pMT/His-V5B vector to obtain a plasmid for expression of His-V5-tagged hEGFR-501 ectodomain sequences.

hEGFR ectodomain fused with U region (referred to as hEGFR-448+U or EBIP): EBIP was synthesized by fusing the U region from ERRP to hEGFR ectodomain (referred to as hEGFR-448+U or EBIP). The following steps were taken to construct the expression vector.

Step i. hEGFR sequences from amino acids 1 to 448 were first PCR amplified using the following primers: (forward) 5′-CGCAAGCTTCGGGAGAGCCGGAGCGAGC-3′ (SEQ ID NO: 5) and (reverse) 5′-CGCGTTAACGATGTTATGTTCAGGCT-3′ (SEQ ID NO: 6). This PCR product was digested with HindIII and HpaI and gel purified for subsequent three-way ligation.

Step ii. The U region epitope from ERRP was synthesized as oligonucleotides with codons optimized for human expression. The following oligonucleotides were used:

Oligo-1,
(SEQ ID NO: 7)
5′-
AGCGCGGCGCCGTGGCAGGTTCCGTCTCTTTCTTGGCAGGCCGTTACCAG
GCCG-3′;
Oligo-2,
(SEQ ID NO: 8)
5′-
CTGGTAACGGCCTGCCAAGAAAGAGACGGAACCTGCCACGGCGCCGCG-
3′;
Oligo-3,
(SEQ ID NO: 9)
5′-
CTTCATCCGCTAGCCCAAAACCGCGTCAGCTGGGACACAGGCCCCTCTAG
ACGC-3′;
and
Oligo-4,
(SEQ ID NO: 10)
5′-
CCGCGTCTAGAGGGGCCTGTGTCCCAGCTGACGCGGTTTTGGGCTAGCGG
ATGAAGCGGC-3′.

The oligonucleotides were phosphorylated at the respective 5′ ends using T4 polynucleotide kinase and annealed as follows: oligos 1+2 and oligos 3+4. The annealed products were ligated to obtain a contiguous U region sequence. This double-stranded U region sequence was then used as template in a PCR using the following primers: (forward) 5′-AGCGCGGCGCCGTGGCAG-3′ (SEQ ID NO: 11) and (reverse) 5′-CCGCGTCTAGAGGGGCCT-3′ (SEQ ID NO: 12). The PCR product was cut with a combination of SfoI and XbaI restriction enzymes, and the product was gel purified.

Step iii. The PCR-amplified products from steps i and ii were ligated into HindIII plus XbaI cut vector plasmid pcDNA-3/myc-His-A to obtain a recombinant plasmid for expression of myc-His-tagged hEGFR+U protein.

Step iv. The cDNA insert of the recombinant plasmid from step iii above was PCR amplified using the forward primer from step i and the reverse primer from step ii. The PCR product was then cut with XbaI and ligated into EcoRV plus XbaI cut pMT/V5-HisA vector (Invitrogen) to obtain a construct for expression of V5/His-tagged hEGFR+U protein. The V5 and 6×His tags are located at the COOH-terminal end of the plasmid (just after the U region).

Rat ERRP. Rat ERRP has been described previously and detailed in U.S. Pat. Nos. 6,399,743 and 6,582,934 (GenBank accession number AF187818). It is composed of 478 amino acids.

All the constructs were sequenced to confirm the validity of the inserts.

Generation of recombinant EBIP included the following aspects. Recombinant EBIP was generated using the Drosophila expression system, as described for ERRP by Marciniak et al. (Marciniak D J, Moragoda L, Mohammad R M, et al. Epidermal growth factor receptor-related protein: a potential therapeutic agent for colorectal cancer. Gastroenterology 2003; 124:1337-47). In brief, expression vector pMT/V5-HisA containing the entire reading frame of ERRP, rEGFR-448, hEGFR-501, or EBIP cDNA was transfected into Drosophila S2 cells with pCoHygro plasmid (Invitrogen), which confers hygromycin resistance. The stable cell line was induced with 0.5 mmol/L CuSO₄ to express respective fusion protein. Proteins were purified from the crude cell lysate using polyhistidine antibodies (Invitrogen) conjugated to Sepharose 4B (Pharmacia). The activity of ERRP/EBIP was determined by MTT assay. ERRP/EBIP with at least 80% growth-inhibitory effect was selected for all experiments.

Growth inhibition assay included the following aspects. Cell growth was determined by MTT assay (Nautiyal J, Majumder P, Patel B B, Lee F Y, Majumdar A P. Src inhibitor dasatinib inhibits growth of breast cancer cells by modulating EGFR signaling. Cancer Lett 2009). Briefly, 5,000 cells per well were treated in 96-well culture plates for 24 or 48 hours in the absence (control) or presence of affinity-purified EBIP and/or dasatinib, as described in the figure legends, with six replicates. At the end of the treatment period, cells were incubated with 10% of 5 mg/mL stock of MTT and incubated for 3 hours at 37° C.

Analysis of interaction between two drugs included the following aspects. Combination index (CI) method adapted for in vitro anticancer drug testing was used to determine the nature of interaction between the two agents as described by Majumdar APN, Banerjee S, Nautiyal J, et al. Curcumin synergizes with resveratrol to inhibit colon cancer. Nutr Cancer 2009; 61:544-53. Based on CI values, the extent of synergism/antagonism may be determined. In general, CI<1 suggests synergy, whereas CI>1 indicates antagonism between the drugs. CI values in the range of 0.9 to 1.10 suggest mainly additive effects of the drugs, those between 0.9 and 0.85 would suggest slight synergy, and values in the range of 0.7 to 0.3 are indicative of moderate synergy. Any value <0.3 will suggest strong synergistic interactions between the drugs.

Western blot analysis included the following aspects. Western blot analysis was done as described by Nautiyal J, Majumder P, Patel B B, Lee F Y, Majumdar A P. Src inhibitor dasatinib inhibits growth of breast cancer cells by modulating EGFR signaling. Cancer Lett 2009. Briefly, aliquots of cell lysates containing 80 μg protein were separated by SDS-PAGE. Electrophoresed proteins were transferred onto nitrocellulose membranes and detected using specific primary and secondary antibodies. The protein bands were visualized by enhanced chemiluminescence detection kit (Amersham Biosciences/Amersham Pharmacia Biotech). The membranes were reprobed for β-actin as loading control. All Western blots were done at least thrice for each experiment.

Assessment of apoptosis included the following aspects.

DNA histone fragmentation ELISA: Approximately 1×10⁵ cells per well were plated and treated the same way as described above. After 24 hours, the cells were lysed, and apoptosis was determined using the Cell Death Detection ELISA^{PLUS} kit from Roche Diagnostics.

Acridine orange staining: The cells were treated and collected as described for DNA fragmentation assay. They were washed once with cold 1×PBS and resuspended in 1×PBS (0.5×10⁶ to 1×10⁶ cells/mL). Cell suspension (50 μl) was stained with 50 μL of acridine orange/ethidium bromide mixture according to the manufacturer's instructions. Within 5 minutes of addition of the acridine orange/ethidium bromide mixture, 10 μL aliquots containing 300 to 500 cells were counted under a fluorescent microscope. Cells that stained positive for acridine orange fluoresced green (live cells), whereas cells that stained positive for ethidium bromide fluoresced red (dead cells). Results were calculated as (ethidium bromide-stained cells/ethidium bromide- or acridine orange-stained cells)×100.

Tyrosine kinase assay included the following aspects. EGFR kinase activity was determined using the Chemicon Assay kit essentially according to the manufacturer's instructions. Briefly, MDA-MB-468 cells were treated for 24 hours with dasatinib and/or EBIP. At the end of the treatment period, cells were collected and lysed and aliquots of 500 μg protein were subjected to immunoprecipitation with anti-EGFR antibody (Santa Cruz Biotechnology) as described by Marciniak D J, Moragoda L, Mohammad R M, et al. Epidermal growth factor receptor-related protein: a potential therapeutic agent for colorectal cancer. Gastroenterology 2003; 124:1337-47. After overnight incubation at 4° C., the lysates were centrifuged and the Sepharose beads were washed thrice with lysis buffer. Subsequently, the immunobeads were assayed for kinase activity. The samples were read at 450 nm, and the results were presented as relative to untreated control.

Severe combined immunodeficient mice xenografts of MDA-MB-468 cells included the following aspects. Four-week-old female ICR/severe combined immunodeficient (SCID) mice, obtained from Taconic Laboratory, were s.c. injected with 10×10⁶ MDA-MB-468 breast cancer cells (Marciniak D J, Moragoda L, Mohammad R M, et al. Epidermal growth factor receptor-related protein: a potential therapeutic agent for colorectal cancer. Gastroenterology 2003; 124:1337-47). When tumor burden reached 1,500 to 2,000 mg, mice were euthanized. The tumors were removed, cut into 20 to 30 mg fragment, and subsequently transplanted (s.c.) bilaterally into similarly conditioned 28 animals. Once palpable tumors were formed, animals were randomly divided into four groups: control (vehicle), dasatinib (10 mg/kg, every other day gavage), EBIP (s.c.; 25 μg/mouse, every other day), and dasatinib+EBIP. Treatment was started on day 7 and continued until day 23. Animals and tumor burden were followed for up to 55 days. Tumor measurements were carried out at multiple time points during the experimental period. Mice were weighed and monitored for signs of toxicity. Tumor weights in SCID mice were estimated as tumor weight (mg)=(A×B2)/2, where A and B are the tumor length and width (in mm), respectively. At the end of the experiments, the mice were sacrificed and the residual tumors were harvested for Western blot analysis and fixed in buffered formalin and processed for immunohistochemistry as described by Marciniak D J, Moragoda L, Mohammad R M, et al. Epidermal growth factor receptor-related protein: a potential therapeutic agent for colorectal cancer. Gastroenterology 2003; 124:1337-47 and Patel B B, Yu Y, Du J, Levi E, Phillip P A, Majumdar A P. Age-related increase in colorectal cancer stem cells in macroscopically normal mucosa of patients with adenomas: a risk factor for colon cancer. Biochem Biophys Res Commun 2009; 378:344-7.

Immunohistochemical analysis included the following aspects. For immunohistochemical staining, an immunoperoxidase method was used with a streptavidin-biotinylated horseradish peroxidase complex (Dako) as described by Marciniak D J, Moragoda L, Mohammad R M, et al. Epidermal growth factor receptor-related protein: a potential therapeutic agent for colorectal cancer. Gastroenterology 2003; 124:1337-47 and Patel B B, Yu Y, Du J, Levi E, Phillip P A, Majumdar A P. Age-related increase in colorectal cancer stem cells in macroscopically normal mucosa of patients with adenomas: a risk factor for colon cancer. Biochem Biophys Res Commun 2009; 378:344-7. Briefly, sections of formalin-fixed, paraffin-embedded tissue blocks were deparaffinized and rehydrated in 0.1 mol/L PBS (pH 7.4), treated for 15 minutes with blocking serum (Vector Laboratories), and subsequently incubated overnight in a refrigerated humidity chamber with antibodies to either anti-V5 or phospho-EGFR (Tyr1173). The next day, the slides were washed thrice in 1×PBS and incubated with a biotin-conjugated secondary antibody at room temperature for 30 minutes and finally with peroxidase-conjugated streptavidin at room temperature for 30 minutes. Peroxidase activity was detected with the enzyme substrate 3-amino-9-ethylcarbazole. For negative controls, sections were treated in the same way except that they were incubated with 1×TBS instead of the primary antibody. All slides were cover-slipped and examined under a 10× objective.

Determination of apoptosis by TUNEL assay included the following aspects. Paraffin-embedded tissues were sectioned as described, and the TUNEL assay was done to detect apoptotic cells using the In Situ Cell Death Detection kit (Roche Applied Science) according to the manufacturer's instructions as described by Marciniak D J, Moragoda L, Mohammad R M, et al. Epidermal growth factor receptor-related protein: a potential therapeutic agent for colorectal cancer. Gastroenterology 2003; 124:1337-47 and Levi E, Mohammad R, Kodali U, et al. EGF-receptor related protein causes cell cycle arrest and induces apoptosis of colon cancer cells in vitro and in vivo. Anticancer Res 2004; 24:2885-91. 3-Amino-9-ethylcarbazole was used as chromogen, and the sections were counterstained with hematoxylin. Apoptotic cell nuclei appeared as red-stained structures against a blue-violet background. The apoptotic cells within each section were counted with a 10× objective.

Statistical analysis included the following aspects. Unless otherwise stated, data were expressed as mean±SD. Where applicable, the results were compared by using the unpaired, two-tailed Student's t test, as implemented by Excel 2000 (Microsoft Corp.). P values of <0.05 were considered statistically significant. One-way ANOVA, SPSS 10.0, was applied for analysis of in vivo data.

The experiments generated the following results.

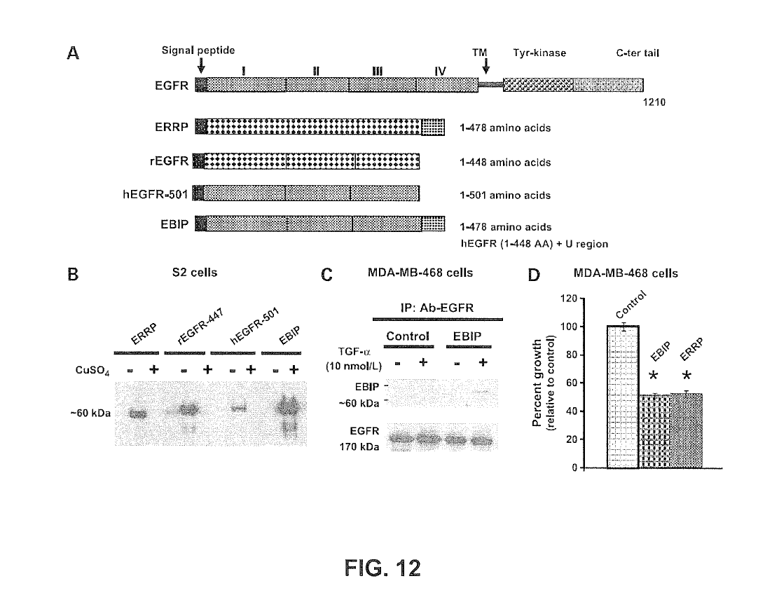

Generation and characterization of EBIP included the following aspects. FIG. 12, panel A depicts a schematic representation of four different plasmid constructs that were generated. They are (ii) full-length rat ERRP (composed of 478 amino acids plus the U region) that we generated earlier, (iii) rat ERRP (composed of 1-448 amino acids) that lacked the U region (referred to as rEGFR-448; ref. 14), (iv) hEGFR ectodomain that contained 1 to 501 amino acids (referred to as hEGFR-501), and (v) hEGFR ectodomain that contained 1 to 448 amino acids plus the U region (hEGFR-448+U; also referred to as EBIP). A schematic representation of hEGFR is also depicted in FIG. 12A (i).

Western blot analysis of Drosophila S2 cells lysates using antihistidine antibodies revealed a marked stimulation in synthesis of the respective recombinant protein following incubation with 0.5 mmol/L CuSO₄ for 24 hours (FIG. 12B). In the absence of 0.5 mmol/L CuSO₄, no expression of EBIP was detected (FIG. 12B). Because EBIP contains the ligand binding ectodomain of hEGFR, we postulated that it will sequester the ligand, leading to heterodimerization with members of the EGFRs. However, such heterodimers, as has been reported for ERRP and EGFR, would likely be inactive because ERRP is devoid of the cytoplasmic domain. Indeed, when MDA-MB-468 cells containing high levels of EGFR were preincubated with EBIP, followed by induction with TGF-α, we found EBIP to coimmunoprecipitate with EGFR, whereas in the absence of TGF-α (control) no EBIP band could be detected (FIG. 12C).

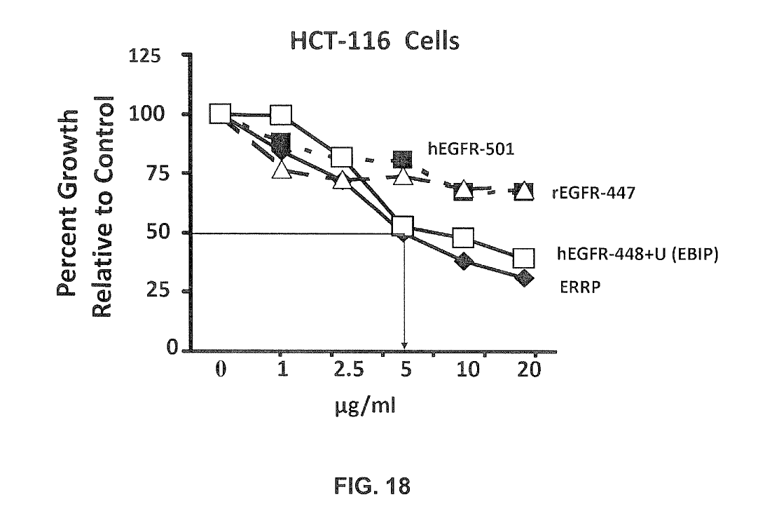

Additionally, growth-inhibitory activity of EBIP was compared with ERRP in human breast cancer cells. Both ERRP and EBIP were found to be equally effective in inhibiting the growth of MDA-MB-468 cells (FIG. 12D). We also compared the growth-inhibitory properties of hEGFR-501, hEGFR-448+U, ERRP, and rEGFR-447 (lacked U region) in colon cancer HCT-116 cells (FIG. 18). We observed that whereas ERRP or EBIP at a dose of 20 μg/mL caused a marked 70% inhibition of growth of HCT-116 cells, the same dose of hEGFR-501 or rEGFR-447 produced only a small 20% to 25% inhibition in cellular growth when compared with the corresponding controls (FIG. 18). The results suggest that the U region is important for the growth-inhibitory properties of ERRP and EBIP.

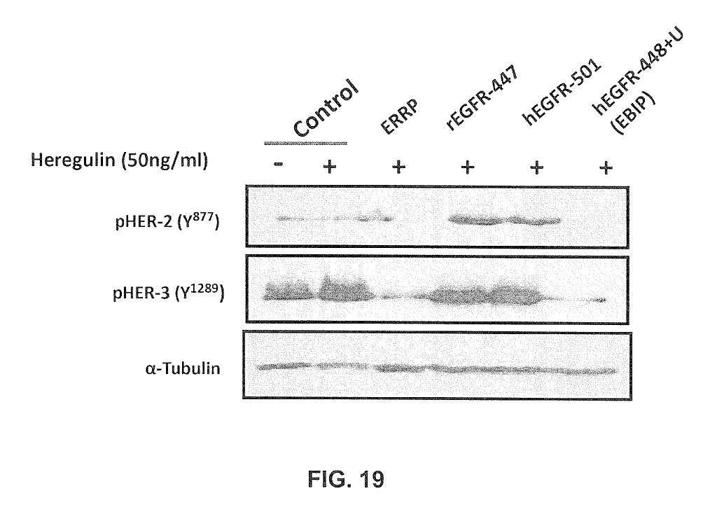

ERRP is a pan-ErbB inhibitor that targets multiple members of the EGFR family (Xu H, Yu Y, Marciniak D, et al. Epidermal growth factor receptor (EGFR)-related protein inhibits multiple members of the EGFR family in colon and breast cancer cells. Mol Cancer Ther 2005; 4:435-42). As shown below, EBIP also inhibited the growth of different breast cancer cells that express varying levels of EGFR and its family members, indicating potential pan-ErbB nature of this protein. Whereas both ERRP and EBIP were able to inhibit heregulin-induced activation of HER-2 and HER-3 in MDA-MB-453 breast cancer cells, neither rEGFR-447 nor hEGFR-501 was effective in this matter (FIG. 19). Taken together, the results suggest a role for the U region of ERRP in eliciting the growth-inhibitory properties of ERRP and EBIP.

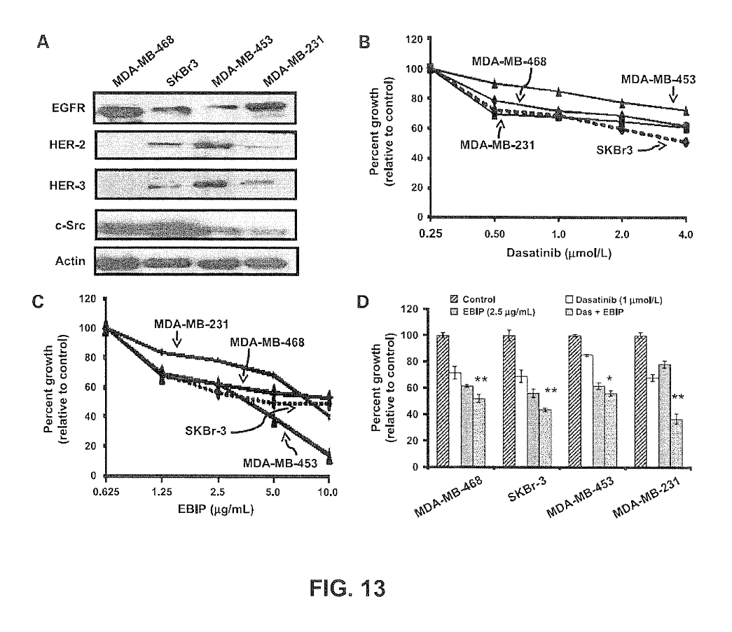

EBIP synergizes with dasatinib to inhibit the growth of human breast cancer cells. In the first set of experiments, effects of EBIP and dasatinib, each alone or in combination, on the growth of four different breast cancer cells expressing F2 varying levels of EGFRs (FIG. 13A) was examined. Both dasatinib and EBIP were effective in inhibiting the growth of all four breast cancer cells (FIGS. 13B and 13C); whereas dasatinib caused a 20% to 40% growth inhibition among different cell lines, EBIP produced a 40% to 90% of the same. When dasatinib (1 μmol/L) and EBIP (2.5 μg/mL) were combined, the magnitude of inhibition of growth was greater than either of the agent alone (FIG. 13D), indicating a greater effectiveness of the combination therapy than monotherapy.

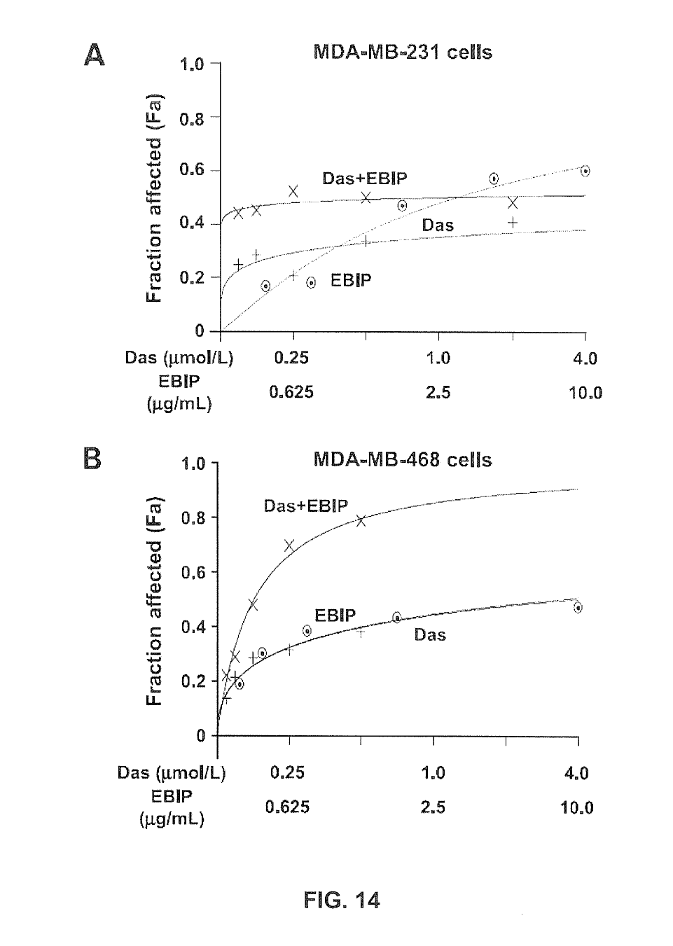

To determine the nature of interactions between EBIP and dasatinib, synergy analysis was done with two triplenegative breast cancer cell lines: MDA-MB-231 and MDAMB-468. The results of the dose response were analyzed using CalcuSyn software (Biosoft). They show that the combination therapy is superior to monotherapy in both breast cancer cell lines (FIGS. 14A and 14B). The fraction of cells affected in response to each treatmentwas further used to do synergy analysis with CalcuSyn. The CI, <1.0, which suggests a synergistic interaction between the two agents, was noted for all the combination doses for both breast cancer cell lines (Table 1 and Table 2). Taken together, the results suggest that EBIP acts synergistically with dasatinib. In all subsequent experiments, dasatinib at a dose of 1 μmol/L and EBIP at a concentration of 2.5 μg/mL were used in MDA-MB-468 cells. The rationale for using MDA-MB-468 cells is that they express only EGFR, which will result in the formation of homodimers in response to ligand induction.

TABLE 1
Calcusyn analysis reveals synergistic interactions
between dasatinib and EBIP in MDA-MB-231 cells.

| Dasatinib | | EBIP | | Dasatinib + EBIP | |
|---|---|---|---|---|---|
| (μM) | (Fa) | (μg/ml) | (Fa) | (Fa) | (CI) |
| 0.50 | 0.354 | 1.25 | 0.105 | 0.462 | 0.076 |
| 1.0 | 0.322 | 2.5 | 0.223 | 0.506 | 0.128 |
| 2.0 | 0.353 | 5.0 | 0.387 | 0.151 | 0.247 |
| 4.0 | 0.392 | 10.0 | 0.535 | 0.544 | 0.447 |

Combination Index (CI) < 1.0 suggests synergy; abbreviations: Fa, fraction affected; CI, confidence interval.

TABLE 2
Calcusyn analysis reveals synergistic interactions
between dasatinib and EBIP in MDA-MB-468 cells.

| Dasatinib | | EBIP | | Dasatinib + EBIP | |
|---|---|---|---|---|---|
| (μM) | (Fa) | (μg/ml) | (Fa) | (Fa) | (CI) |
| 0.50 | 0.216 | 1.25 | 0.302 | 0.289 | 0.714 |
| 1.0 | 0.286 | 2.5 | 0.385 | 0.480 | 0.249 |
| 2.0 | 0.315 | 5.0 | 0.435 | 0.698 | 0.070 |
| 4.0 | 0.382 | 10.0 | 0.475 | 0.789 | 0.051 |

Combination Index (CI) < 1.0 suggests synergy; abbreviations: Fa, fraction affected; CI, confidence interval.

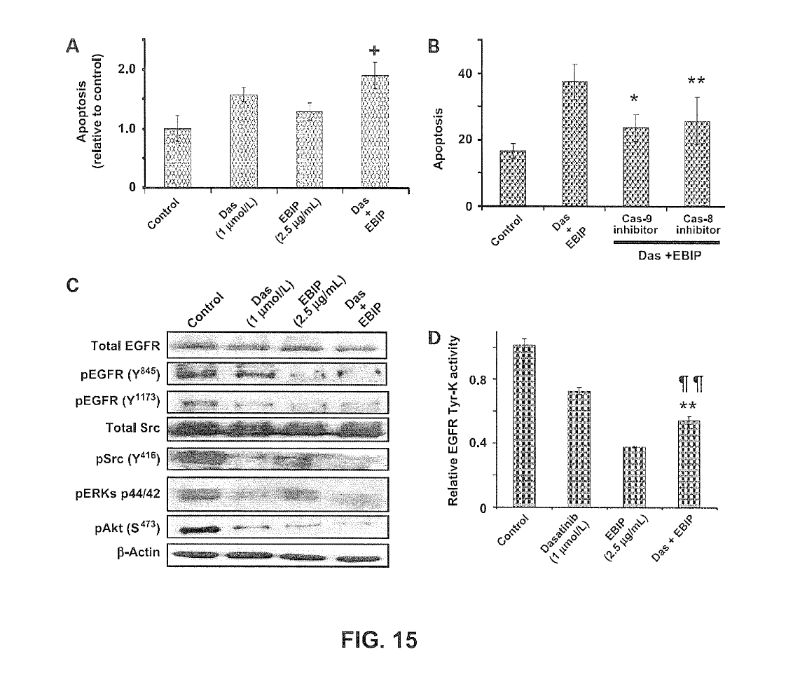

EBIP and/or dasatinib induce apoptosis and inhibit tyrosine kinase activity. The combined therapy was further tested for its efficacy for induction of apoptosis, which was found to be more effective in MDA-MB-468 cells than either agent alone (FIG. 15A). To further identify the apoptotic pathways, specific inhibitors of caspase-8 (Z-IETD-FMK) and caspase-9 (Z-LEHD-FMK) were used. The cells were preincubated with specific inhibitors of caspase-8 or caspase-9 for 3 hours, subsequently exposed to the combination of EBIP and dasatinib. In the absence of the inhibitors, the combined therapy caused significant apoptosis. However, the addition of specific caspase inhibitor(s) blocked apoptosis induction by the combined therapy, indicating the activation of respective caspase(s) in response to the treatment. This suggests the involvement of both intrinsic (caspase-9) and extrinsic (caspase-8) pathways of apoptosis (FIG. 15B).

EBIP and dasatinib, each alone, inhibited the phosphorylation of EGFR and c-Src, respectively, in MDAMB-468 cells (FIG. 15C). Again, the combination therapy was much more effective than either agent alone in inhibiting activation of EGFR, c-Src, as well as downstream targets Akt and mitogen-activated protein kinase (FIG. 15C). Dasatinib and/or EBIP inhibited EGFR phosphorylation/activation at both transphosphorylation (Tyr⁸⁴⁵, mediated by c-Src) and autophosphorylation (Tyr¹¹⁷³) sites. Although dasatinib and EBIP inhibit signaling differentially, the combination therapy, as expected, provided a better therapeutic benefit in achieving a greater inhibition of downstream signaling events (FIG. 15C). Likewise, EGFR tyrosine kinase activity was greatly inhibited by the combined therapy (FIG. 15D). At this time, the slight increase in tyrosine kinase activity in response to the combined therapy is not properly understood. This may be due to the involvement of compensatory mechanisms as reported for signal transducer and activator of transcription 3 (STAT3) in response to dasatinib in head and neck cancer and mesothelioma.

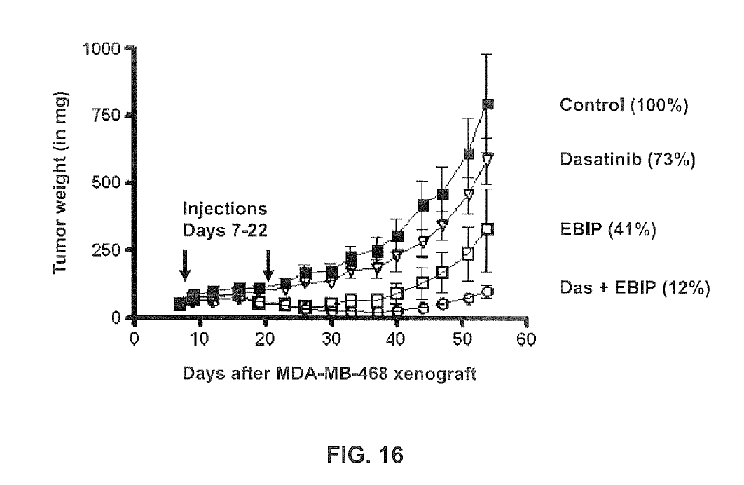

Combined therapy is more effective in inhibiting the growth of breast cancer cell-derived xenografts in SCID mice. The objective of this experiment was to examine the effectiveness of monotherapy versus combination therapy on tumor growth. None of the treatments caused any significant change in body weight, indicating no apparent toxicity (data not shown). With respect to tumor growth, dasatinib produced no significant inhibition, whereas EBIP and the combination therapy significantly reduced tumor growth, suggesting effectiveness of the combination therapy (P<0.05; FIG. 16; Table 3). The results show that whereas dasatinib and EBIP, each alone, caused about 27% and about 59% inhibition, combination therapy produced a marked (about 90%) suppression of tumor growth when compared with the vehicle-treated controls (FIG. 16; Table 3). ANOVA analysis shows that the differences among the groups are significant and the possibility of the results assuming null hypothesis is 0.003 (P<0.05; Table 4). More importantly, the data show that growth of the tumor in the combination treatment group was minimal 32 days after treatment. At this time, the tumor volume was only about 12% of the vehicle-treated control (FIG. 16).

TABLE 3
Breast cancer xenografts weight (in mg) among different
groups at the end of in vivo investigations (day-55).

| | Study Group tumor weight at the end of 55 days | | | |
|---|---|---|---|---|
| | | | | Dasatinib + |
| | Control | Dasatinib | EBIP | EBIP |
| | (n = 5) | (n = 5) | (n = 4) | (n = 6) |
| Mean ± SD | 795 ± 406 | 584 ± 189 | 326 ± 304 | 98.5 ± 59.2 |
| 95% CI for | 546.4-1043 | 335.6-832.0 | 48.77-603.7 | 128.1-325.1 |
| mean | | | | |

n = no. of mice in the group; SD = standard deviation; CI = confidence interval.

TABLE 4
Results of one-way ANOVA statistical test for the in vivo study.

| Source of variation | Sum of Squares | df | Mean squares | F |
|---|---|---|---|---|
| Between | 1.48E+06 | 3 | 4.94E+05 | 7.22 |
| Error | 1.09E+06 | 16 | 6.85E+04 | |
| Total | 2.58E+06 | 19 | | |

NOTE:
The probability of this result, assuming the null hypothesis, is 0.003. Abbreviations: df, degree of freedom.

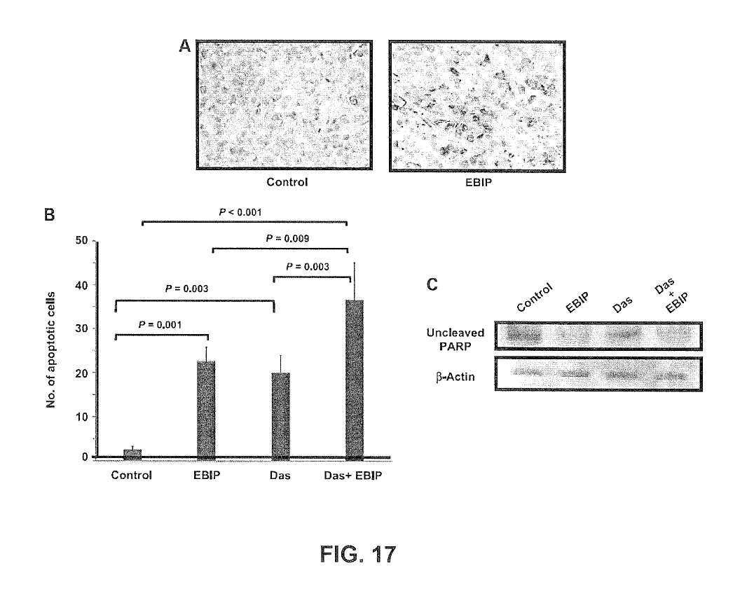

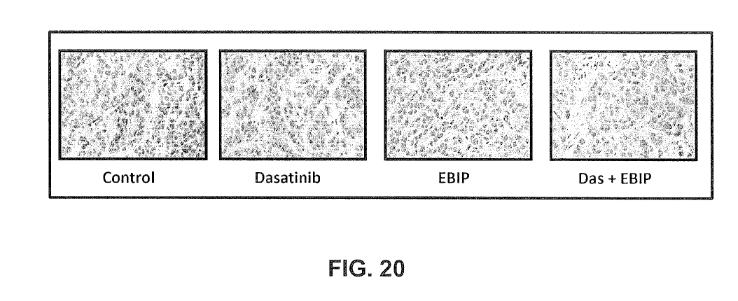

The animals were sacrificed at the end of the 55-day experimental period. To determine whether EBIP reaches the tumor, the tissues are analyzed for the presence of EBIP. Indeed, significant expression of EBIP was observed in the tumors of EBIP-treated mice (FIG. 17A). To determine whether inhibition of tumor growth in SCID mice could be the result of increased apoptosis, a TUNEL assay was conducted and PARP cleavage examined in the tumors. As expected, the combined therapy caused a marked induction of apoptosis as evidenced by the increased number of apoptotic cells and PARP (FIGS. 17B and 17C). The tumors were analyzed for relative abundance of phospho-EGFR by immunohistochemistry using anti-phospho-EGFR (Tyr¹¹⁷³) antibodies. Tumor remnants from mice treated with EBIP or EBIP+dasatinib showed no detectable immunoreactivity for phospho-EGFR, whereas those from the controls and dasatinib-treated mice showed the presence of phospho-EGFR (FIG. 20). However, the intensity of phospho-EGFR immunoreactivity in tumors from dasatinib-treated mice was weaker than those from the controls (FIG. 20).

Interference with activation of EGFR and/or its family members represents a promising strategy for the development of targeted therapies against a wide variety of epithelial cancers because of their preponderance in a variety of neoplastic cells. Indeed, several inhibitors of EGFRs have been developed to interrupt the intracellular signaling induced by activation of EGFR. Small-molecule inhibitors of EGFR, gefitinib (Iressa) and erlotinib (Tarceva), approved by the Food and Drug Administration, have now been used for treatment of many epithelial cancers, including breast cancer, but with limited success. Although monoclonal antibodies against EGFR (cetuximab) and HER-2 (trastuzumab) showed signs of success in a limited number of patients with tumors that expressed high levels of EGFR or HER-2, failure in others may partly be due to the fact that most solid tumors express more than one member of the EGFR family, and coexpression of multiple EGFR family members leads to an enhanced transforming potential and worsened prognosis. Therefore, identification of inhibitor(s), targeting multiple members of the EGFR family, is likely to provide a therapeutic benefit to a broad range of patient population.

The data suggests that EBIP, as has been reported for ERRP, is a potential pan-ErbB inhibitor targeting multiple members of the EGFR family. This inference is supported by the observation that EBIP inhibits the growth of several breast cancer cells that express varying levels of different EGFRs. EBIP forms a heterodimer with EGFR in MDA-MB-468 cells, resulting in decreased EGFR signaling. The fact that daily administration of EBIP leads to a significant reduction in the growth of SOD mice xenografts of breast cancer MDA-MB-468 cells, which express very high levels of EGFR and little or no other ErbBs, further corroborates the postulation that EBIP could be used to inhibit growth of EGFR-expressing tumors. This and the fact that EBIP also inhibits growth of several other breast cancer cells that express other members of the EGFR family and also inhibits heregulin-induced activation of HER-2 and HER-3 in breast cancer cells suggest that EBIP, as has been reported for ERRP, could potentially be a pan-ErbB inhibitor.

Although the precise mechanisms by which EBIP inhibits activation of EGFR and its family members and in turn cellular growth are not fully understood, studies with ERRP suggest that this peptide, which is structurally and functionally similar to EBIP, inhibits EGFR function by sequestering EGFRs ligand(s), leading to heterodimerization with one of the EGFR family members, which is functionally inactive. A similar phenomenon may be responsible for the growth-inhibitory properties of EBIP because EBIP contains the ligand binding domain of EGFR. The possibility that ectodomains of EGFR inhibit EGFR signaling by sequestering their ligands comes from the observation by Garrett et al. (Garrett T P, McKern N M, Lou M, et al. Crystal structure of a truncated epidermal growth factor receptor extracellular domain bound to transforming growth factor α. Cell 2002; 110:763-73) that a truncated EGFR with only three of the four extracellular subdomains binds EGF and TGF-α with at least 10-fold higher affinity than the full-length extracellular domain of EGFR, rendering them unavailable for binding to and activation of receptors. Because EBIP, like ERRP, lacks most of the extracellular domain IV, it is reasonable to predict that EBIP will also be effective in preferentially binding/sequestering ligands of EGFR. The data supports this contention in that EBIP coimmunoprecipitated with EGFR after induction with TGF-α.

In addition to EGFRs, aberrant activation of c-Src has been observed in many solid tumors, including breast cancers. Furthermore, co-overexpression of EGFRs and c-Src is associated with higher incidence of metastasis and poor survival. Because of the involvement of Src in the development and progression of many solid tumors, several Src inhibitors, including dasatinib, have been tested in solid tumors but with limited success. This could partly be due to the presence and dominance of compensatory pathways in the cancer cells. For instance, STAT3 pathway is inhibited by dasatinib transiently and through a compensatory pathway and is reactivated as early as 24 hours. It has been suggested that STAT3 inhibitors show synergistic interactions with dasatinib in head and neck squamous cell carcinoma. Therefore, to achieve a better therapeutic efficacy, targeting multiple pathways simultaneously is warranted. The observation that dasatinib together with EBIP causes greater inhibition of growth of breast cancer cells in vitro and in vivo supports the postulation that simultaneous targeting of multiple signaling pathways is an effective therapeutic strategy, demonstrating the effectiveness of a combination therapy of EGFR and Src inhibitors in breast cancer.

The data presented herein show that EBIP is a potential pan-ErbB inhibitor with antitumor activity, EBIP synergizes with dasatinib to suppress growth of several breast cancer cells expressing varying levels of EGFRs, and combination therapy is much more effective in inhibiting the growth of breast cancer cell-derived xenografts than monotherapy. In particular, combination therapy of EBIP and dasatinib is a strategy for the treatment of triple-negative breast cancer.

The present methods and compositions can further include an EGFR with a modified ectodomain in combination with various signal transduction inhibitors, such as tyrosine kinase inhibitors and non-receptor tyrosine kinase inhibitors. For example, one or more of the present proteins, homologues, and recombinant constructs comprising an EGFR with a modified ectodomain may be used with one or more signal transduction inhibitors such as cetuximab (Erbitux™), trastuzumab (Herceptin™), gefitinib (Iressa™), erlotinib (Tarceva™), dasatinib (Sprycel™), imatinib, bevacizumab, sorafenib, and sunitinib.

The present EGFRs with a modified ectodomain and/or combinations with one or more inhibitors may be used to treat one or more of the following conditions: breast cancer, chronic myeloid leukemia (CML), gastrointestinal stromal tumors (GIST), non-small cell lung cancer (NSCLC), colorectal cancer (CRC), pancreatic cancer, renal cell cancer, and head and neck cancer.

The following U.S. patents are incorporated herein by reference: U.S. Pat. Nos. 6,399,743, Majumdar, issued Jun. 4, 2002; 6,582,934, Majumdar, issued Jun. 24, 2003; 6,946,543, Ward et al., issued Sep. 20, 2005; and 7,049,410, Majumdar et al., issued May 23, 2006.

The embodiments and the examples described herein are exemplary and not intended to be limiting in describing the full scope of apparatus, systems, and methods of the present technology. Equivalent changes, modifications and variations of some embodiments, materials, compositions and methods can be made within the scope of the present technology, with substantially similar results.

### NON-LIMITING DISCUSSION OF TERMINOLOGY

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

“A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

What is claimed is:

1. A polypeptide comprising an epidermal growth factor receptor (EGFR) ectodomain coupled at the C-terminus to a U-region.

2. The polypeptide of claim 1, wherein the polypeptide comprises rat EGFR-related protein (ERRP) or human ErbB-inhibitory protein (EBIP).

3. The polypeptide of claim 1, wherein the EGFR ectodomain and the U-region are at least 80% homologous to the EGFR ectodomain and U-region from rat or human.

4. The polypeptide of claim 1, wherein the polypeptide comprises an amino acid sequence according to SEQ ID NO: 14 or SEQ ID NO: 16.

5. The polypeptide of claim 1, wherein the polypeptide comprises an amino acid sequence that is at least 80% homologous to SEQ ID NO: 14 or SEQ ID NO: 16.

6. The polypeptide of claim 1, wherein the EGFR ectodomain comprises an ErbB-2/HER2, ErbB-3/HER-3, or ErbB-4/HER-4 ectodomain.

7. A composition comprising:
a polypeptide comprising an epidermal growth factor receptor (EGFR) ectodomain coupled at the C-terminus to a U-region; and
a tyrosine kinase inhibitor.

8. The composition of claim 7, wherein the tyrosine kinase inhibitor comprises cetuximab, trastuzumab, gefitinib, erlotinib, dasatinib, imatinib, bevacizumab, sorafenib, or sunitinib.

9. The composition of claim 7, wherein the polypeptide comprises EBIP and the tyrosine kinase inhibitor comprises dasatinib.

10. The composition of claim 7, further comprising a pharmaceutically acceptable carrier.

11. The composition of claim 10, wherein the composition is formulated for enteral, parenteral, or topical administration.

12. The composition of claim 10, wherein the composition is a solution, suspension, gel, or powder.

13. A nucleic acid encoding a polypeptide comprising an epidermal growth factor receptor (EGFR) ectodomain coupled at the C-terminus to a U-region.

14. The nucleic acid of claim 13, wherein the polypeptide comprises rat EGFR-related protein (ERRP) or human ErbB-inhibitory protein (EBIP).

15. The nucleic acid of claim 13, wherein the EGFR ectodomain and the U-region are at least 80% homologous to the EGFR ectodomain and U-region from rat or human.

16. The nucleic acid of claim 13, wherein the nucleic acid comprises a nucleotide sequence according to SEQ ID NO: 17 or SEQ ID NO: 18.

17. The nucleic acid of claim 13, wherein the nucleic acid comprises a nucleotide sequence that is at least 80% homologous to SEQ ID NO: 17 or SEQ ID NO: 18.

18. The nucleic acid of claim 13, wherein the EGFR ectodomain comprises an ErbB-2/HER2, ErbB-3/HER-3, or ErbB-4/HER-4 ectodomain.

19. The nucleic acid of claim 13, wherein the nucleic acid is comprised by an expression vector.

20. The nucleic acid of claim 19, wherein the vector is a plasmid.

21. A method for inhibiting epidermal growth factor receptor (EGFR) activity in a cell comprising administering a polypeptide to a cell expressing EGFR or an EGFR family member, the polypeptide comprising an EGFR ectodomain coupled at the C-terminus to a U-region.

22. The method according to claim 21, wherein the polypeptide comprises rat EGFR-related protein (ERRP) or human ErbB-inhibitory protein (EBIP).

23. The method according to claim 21, wherein the EGFR ectodomain and the U-region are at least 80% homologous to the EGFR ectodomain and U-region from rat or human.

24. The method according to claim 21, wherein the polypeptide comprises an amino acid sequence according to SEQ ID NO: 14 or SEQ ID NO: 16.

25. The method according to claim 21, wherein the polypeptide comprises an amino acid sequence that is at least 80% homologous to SEQ ID NO: 14 or SEQ ID NO: 16.

26. The method according to claim 21, wherein the EGFR ectodomain comprises an ErbB-2/HER2, ErbB-3/HER-3, or ErbB-4/HER-4 ectodomain.

27. A method for treating a neoplasm comprising administering to a subject having a neoplasm a composition comprising a therapeutically effective amount of a polypeptide comprising an epidermal growth factor receptor (EGFR) ectodomain coupled at the C-terminus to a U-region.

28. The method according to claim 27, wherein the polypeptide comprises rat EGFR-related protein (ERRP) or human ErbB-inhibitory protein (EBIP).

29. The method according to claim 27, wherein the EGFR ectodomain and the U-region are at least 80% homologous to the EGFR ectodomain and U-region from rat or human.

30. The method according to claim 27, wherein the polypeptide comprises an amino acid sequence according to SEQ ID NO: 14 or SEQ ID NO: 16.

31. The method according to claim 27, wherein the polypeptide comprises an amino acid sequence that is at least 80% homologous to SEQ ID NO: 14 or SEQ ID NO: 16.

32. The method according to claim 27, wherein the EGFR ectodomain comprises an ErbB-2/HER2, ErbB-3/HER-3, or ErbB-4/HER-4 ectodomain.

33. The method of claim 27, wherein the composition further comprises a tyrosine kinase inhibitor.

34. The method of claim 33, wherein the tyrosine kinase inhibitor comprises cetuximab, trastuzumab, gefitinib, erlotinib, dasatinib, imatinib, bevacizumab, sorafenib, or sunitinib.

35. The method of claim 33, wherein the polypeptide comprises EBIP and the tyrosine kinase inhibitor comprises dasatinib.

36. The method of claim 27, further comprising a pharmaceutically acceptable carrier.

37. The method of claim 27, wherein the composition is formulated for enteral, parenteral, or topical administration.

38. The method of claim 27, wherein the composition is a solution, suspension, gel, or powder.

39. The method of claim 27, wherein the neoplasm is a breast, colon, ovarian, stomach, renal, pancreatic, bladder, or skin neoplasm.

40. The method of claim 27, wherein the neoplasm is breast cancer, chronic myeloid leukemia (CML), gastrointestinal stromal tumor (GIST), non-small cell lung cancer (NSCLC), colorectal cancer (CRC), pancreatic cancer, renal cell cancer, or head and neck cancer

41. The method of claim 27, wherein the amount is from about 0.1 to about 20 mg/kg.

42. The method of claim 27, wherein the amount is from about 0.5 mg/kg to about 12 mg/kg.

43. The method of claim 27, wherein the amount is from about 1 mg/kg to about 8 mg/kg.

44. The method of claim 27, wherein the method is employed for inhibiting activity of EGFR or an EGFR family member in a tumor cell, for suppressing tumor growth, for reducing the size or extent of a tumor, for delaying development or metastasis of a tumor, for killing tumor cells, or for a combination thereof.
